(19) United States
(12) Patent Application Publication
(10) Pub. No.: US 2012/0276124 A1
(43) Pub. Date: Nov. 1, 2012

(54) MAYTANSINOIDS AND THE USE OF SAID MAYTANSINOIDS TO PREPARE CONJUGATES WITH AN ANTIBODY

(75) Inventors: Hervé BOUCHARD, Thiais (FR); Alain COMMERCON, Vitry-Sur-Seine (FR); Claudia FROMOND, Fleury en Biere (FR); Vincent MIKOL, Charenton-le-Pont (FR); Fabienne PARKER, Antony (FR); Ingrid SASSOON, Villejuif (FR); Daniel TAVARES, Natick, MA (US)

(73) Assignee: SANOFI, Paris (FR)

(21) Appl. No.: 13/434,363

(22) Filed: Mar. 29, 2012

Related U.S. Application Data
(63) Continuation of application No. PCT/IB2010/054417, filed on Sep. 30, 2010.

(30) Foreign Application Priority Data
Oct. 2, 2009 (EP) 09305939.2

(51) Int. Cl. A61K 39/395; A61P 35/00; C07K 17/02; A61K 31/537; C07D 498/18; C07K 1/04

(52) U.S. Cl. 424/178.1; 540/462; 530/391.1; 514/229.5

(57) ABSTRACT

The invention relates to a compound of formula (I):

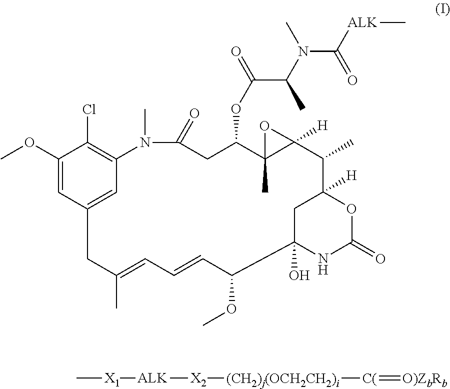

wherein:
ALK is a (C₁-C₆)alkylene group;
X₁ et X₂ are each independently one of the following groups: —CH═CH—, —CO—, —CONR—, —NRCO—, —COO—, —OCO—, —OCONR—, —NRCOO—, —NRCONR′—, —NR—, —S(O)ₙ (n=0, 1 or 2) or —O—;
R and R′ are independently H or a (C₁-C₆)alkyl group;
i is an integer of from 1 to 40, preferably from 1 to 20, and more preferably from 1 to 10;
j is an integer corresponding to 1 when X₂ is —CH═CH— and 2 when X₂ is not —CH═CH—;
Z_{b} is a simple bond, —O— or —NH— and R_{b} is H or a (C₁-C₆)alkyl, (C₃-C₇)cycloalkyl, aryl, heteroaryl or (C₃-C₇)heterocycloalkyl group; or Z_{b} is a single bond and R_{b} is Hal.

### CROSS-REFERENCE TO RELATED APPLICATIONS

This application is a continuation of International application No. PCT/IB2010/054417, filed Sep. 30, 2010, which claims the benefit of priority of European Patent Application No. 09305939.2, filed Oct. 2, 2009, both of which are incorporated herein by reference.

### FIELD

The invention relates to new maytansinoids and the use of said maytansinoids to prepare conjugates with an antibody. The invention also relates to the compositions comprising said maytansinoids and said conjugates.

### BACKGROUND

Many articles have appeared on the attempted specific targeting of tumor cells with monoclonal antibody-drug conjugates (Sela et al, in Immunoconjugates 189-216 (C. Vogel, ed. 1987); Ghose et al. in Targeted Drugs 1-22 (E. Goldberg, ed. 1983); Diener et al. in Antibody mediated delivery systems 1-23 (J. Rodwell, ed. 1988); Pietersz et al, in Antibody mediated delivery systems 25-53 (J. Rodwell, ed. 1988); Bumol et al. in Antibody: mediated delivery systems 55-79 (J. Rodwell, ed. 1988). See also: Monneret C., et al., Bulletin du Cancer 2000, 87(11), 829-38; Ricart A. D., et al., Nature Clinical Practice Oncology 2007, 4, 245-255; Singh R. et Rickson H. K., Therapeutic Antibodies: Methods and Protocols, 2009, 525, 445-467. Different families of cytotoxic agents like taxane derivatives (WO 06061258), leptomycine derivatives (WO 07144709), CC-1065 and analogues (WO 2007102069) or like methotrexate, daunorubicin, doxonrubicin, vincristine, vinblastine, melphalan, mitomycin C, chlorambucil have been used for the conjugation with antibodies.

The use of a targeting antibody having an affinity for the tumor cells makes it possible to deliver the cytotoxic agent directly in the vicinity or directly in the tumor cell, thus increasing the efficiency of the cytotoxic agent while minimizing the side-effects commonly associated with the cytotoxic agents.

Maytansinoids are cytotoxic agents that are derived from maytansin which is a natural product isolated from the cast African shrub Maytenus serrata (U.S. Pat. No. 3,896,111). Many maytansinoids have been prepared; see U.S. Pat. No. 4,151,042; J. Med. Chem. 1978, 21, 31-37; Nature 1977, 270, 721-722, Chem. Pharm. Bull. 1984, 3441-3451; U.S. Pat. No. 4,248,870; U.S. Pat. No. 4,137,230; Chem. Bull. 1984, 3441.

U.S. Pat. No. 5,208,020, U.S. Pat. No. 5,416,064, and R. V. J. Chari, 31 Advanced Drug Delivery Reviews 89-104 (1998) describe conjugates of maytansinoids like L-DM1 (A) or L-DM4 (A′)

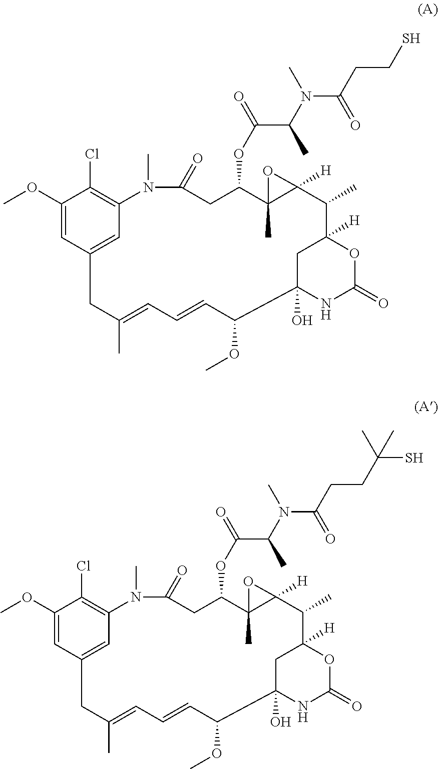

Conjugates of maytansinoids are described in EP 0425235 and WO 2004/103272. In EP 0425235 the following maytansinoids of formulas (B1), (B2) or (B3) are described:

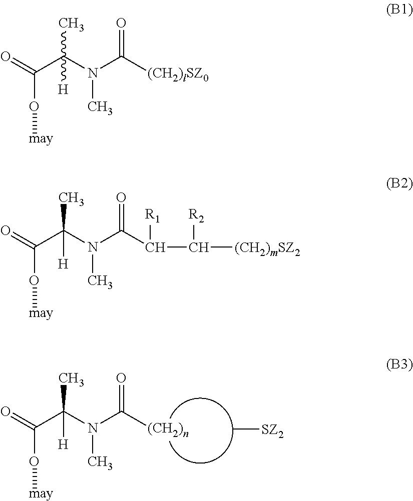

wherein Z₀, Z₁ ou Z₂ represent H or SR.

In WO 2004/103272, maytansinoids of formula (C) are described

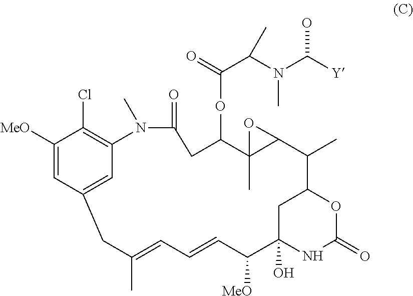

wherein Y′ represents

(CR₇CR₈)ₗ(CR₉═CR₁₀)ₚC≡C_{q}Aᵣ(CR₅CR₆)ₘDₙ(CR₁₁═CR₁₂)ᵣ(C≡C)ₛBₜ(CR₃CR₄)ᵤCR₁R₂SZ

wherein A, B et D represent an optionally substituted cycloalkyl, cycloalkenyl, heteroaryl or heterocycloalkyl group.

In WO 03/068144, compounds of formula (D) are described:

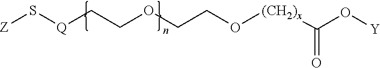

wherein Z is a cytotoxic agent and Q is R₂COO, R₂R₃NCOO, R₂OCOO, R₂O, R₂CONR₃, R₂R₃N, R₂OCONR₃ or S, R₂ is SCR₄R₅R₆. Z may be a maytansinoid derivative chosen among the following ones:

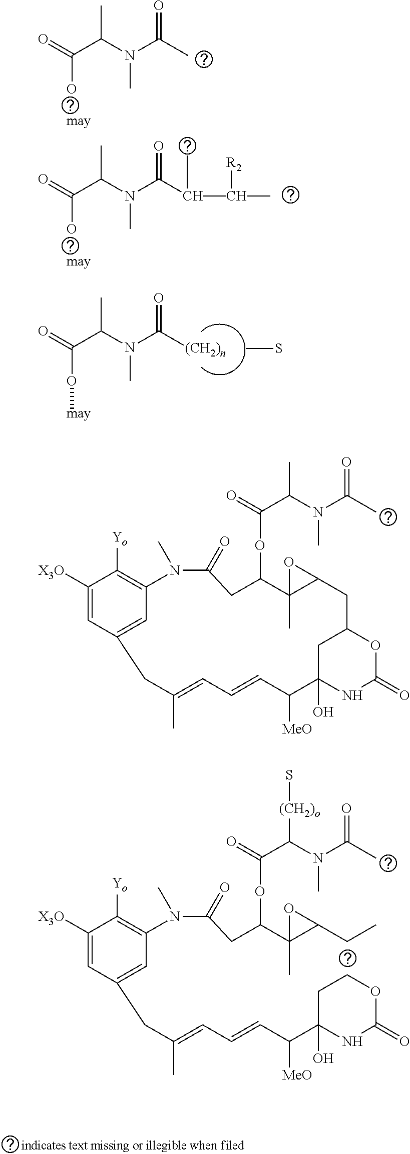

More specifically, the following compounds (D) are disclosed:

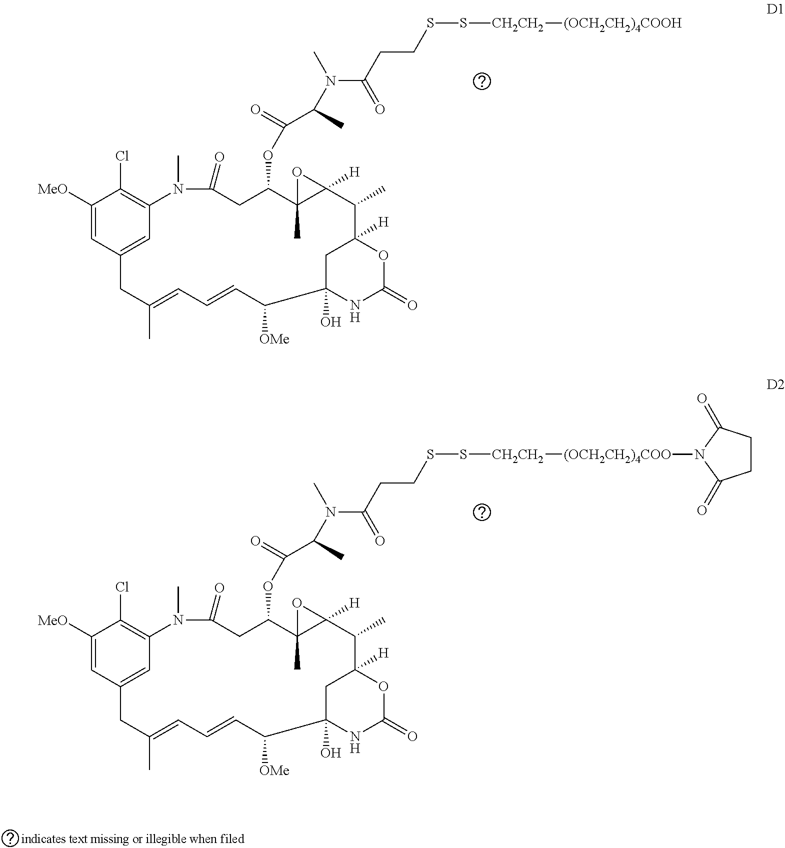

Compounds (D) thus contain an internal disulfide bond in the pegylated linker.

On Dec. 8, 2008, Immunogen Inc. disclosed also at the European Antibody Congress in Geneva conjugates of formula (E):

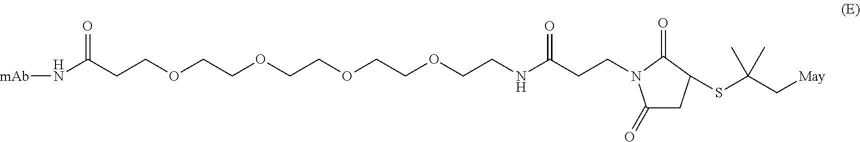

and at the 20^{th} symposium EORTC-NCI-AACR having taken place in October 2008 in Geneva conjugates of formula (E′):

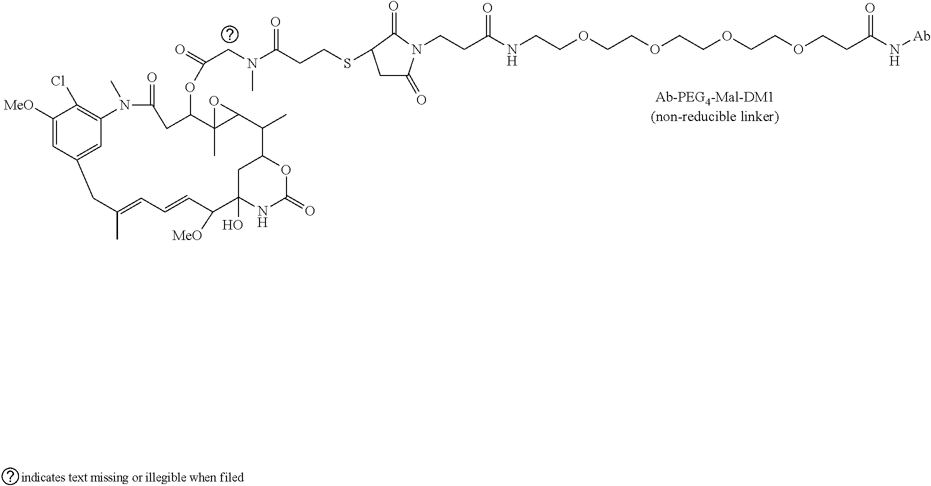

### DEFINITIONS

“Alkyl” means an aliphatic hydrocarbon group which may be straight or branched having 1 to 20 carbon atoms in the chain or cyclic having 3 to 10 carbon atom. Preferred alkyl groups have 1 to 12 carbon atoms in the chain. Exemplary alkyl groups include methyl, ethyl, n-propyl, i-propyl, 2,2-dimethylpropyl, n-butyl, 1-butyl, n-pentyl, 3-pentyl, octyl, nonyl, decyl;
“Cycloalkyl” means a cyclic aliphatic hydrocarbon group having 3 to 10 carbon atom. Preferred cycloalkyl groups have 3 to 8 carbon atoms in the cyclic chain. Exemplary cycloalkyl groups include cyclopropyl, cyclobutyl, cyclopentyl and cyclohexyl;
“Aryl” means an aromatic monocyclic or multicyclic hydrocarbon ring system of 6 to 14 carbon atoms, preferably of 6 to 10 carbon atoms. Exemplary aryl groups include phenyl or naphthyl.
“Heteroaryl” means an unsaturated stable 3 to 14, preferably 5 to 10 membered mono, bi or multicyclic ring wherein at least one member of the ring is a hetero atom. Typically, the heteroatom is, but is not limited to, an oxygen, nitrogen, sulfur, selenium or phosphorus atom. Preferably the heteroatom is an oxygen, nitrogen or sulphur atom. Exemplary heteroaryl groups include pyridyl, pyrrolyl, thienyl, furyl, pyrimidinyl, and triazolyl;
“Heterocycloalkyl” means an cycloalkyl group containing at least one heteroatom wherein at least one member of the ring is a hetero atom
“Alkoxy” means an —O-alkyl group where alkyl is defined as above;
“Alkoyloxy” means an —O—CO-alkyl group where alkyl is defined as above
“Alkylene” means an alkyl group of general formula —CₘH₂ₘ— formed from a straight or branched alkane by removal of two hydrogen atoms. Exemplary alkylene groups include methylene (—CH₂—), ethylene (—CH₂CH₂—), propylene (—CH₂CH₂CH₂—), butylene (—CH₂CH₂CH₂CH₂—), isobutylene

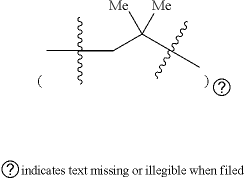

hexylene (—CH₂CH₂CH₂CH₂CH₂CH₂—). A straight alkylene group can be specifically represented by the formula —(CH₂)ₘ—, where m is an integer of from 1 to 20;
“EphA2 receptor” refers to a tyrosine kinase belonging to the Eph receptors family (reviewed in Pasquale, E. B. et al., 2005, Nature Reviews Mol. Cell. Biol., 6, 462-475), and comprising, for example, an amino sequence as in Genbank accession Nos NM_{—}004431 (human EphA2). NM_{—}010139 (murine EphA2), or NXM_{—}345596 (rat EphA2). Human EphA2 is a preferred EphA2 receptor. The term “EphA2 ligand” as used herein refers to a protein that binds to, and optionally activates (e.g. stimulates the autophosphorylation of), an EphA2 receptor. A preferred EphA2 ligand herein is “ephrinA1”, which binds to the EphA2 receptor and comprises, for example, an amino sequence as in Genbank accession NM_{—}004428 (human ephrinA1);
“polyclonal antibody” is an antibody which was produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunized animal
“monoclonal antibody” is an antibody obtained from a population of substantially homogeneous antibodies, i.e. the antibodies forming this population are essentially identical except for possible naturally occurring mutations which might be present in minor amounts. These antibodies are directed against a single epitope and are therefore highly specific;
“naked antibody” is an antibody which is not conjugated to a maytansinoid
“epitope” is the site on the antigen to which an antibody binds. It can be formed by contiguous residues or by non-contiguous residues brought into close proximity by the folding of an antigenic protein. Epitopes formed by contiguous amino acids are typically retained on exposure to denaturing solvents, whereas epitopes formed by non-contiguous amino acids are typically lost under said exposure.

### BRIEF DESCRIPTION OF THE DRAWINGS

FIG. 1 provides a HRMS spectrum after deconvulation of the deglycosylated conjugate of ex. 1.

FIG. 2 provides a HRMS spectrum after deconvulation of the deglycosylated conjugate of ex. 2.

FIG. 3 provides a HRMS spectrum after deconvulation of the deglycosylated conjugate of ex. 3.

FIG. 4 provides a HRMS spectrum after deconvulation of the deglycosylated conjugate of ex. 4.

FIG. 5 provides a HRMS spectrum after deconvulation of the deglycosylated conjugate of ex. 5.

FIG. 6A-6C provides sequences for certain SEQ ID NOS.

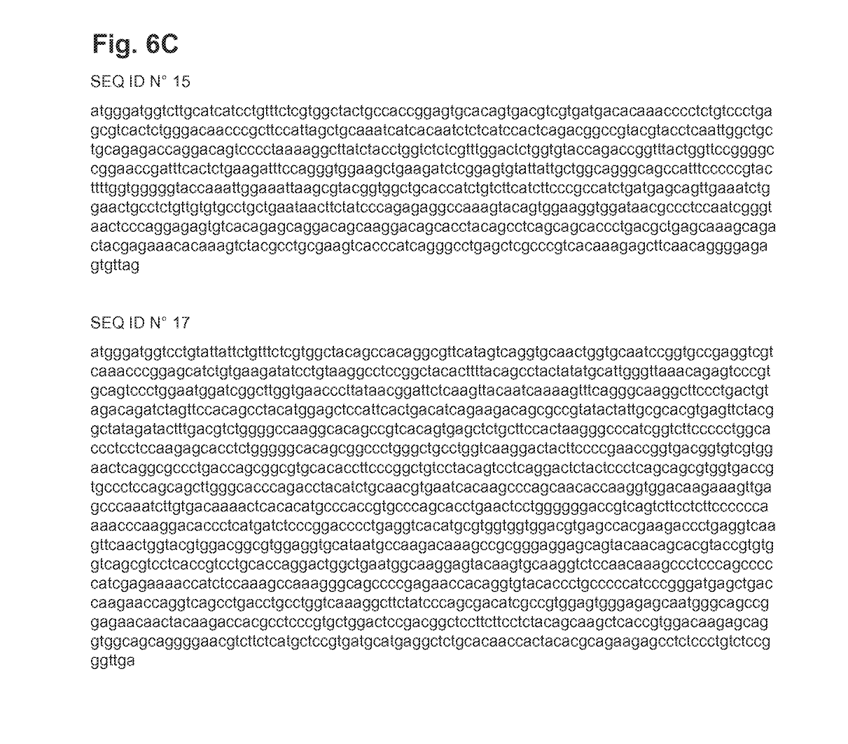

FIG. 7 shows PK parameters for hu2H11R35R74 conjugate at various DARs, providing a bar graph representation of the exposure to (AUC(0-inf); left) and clearance (C1; right) of several conjugates as a function of the DAR after a single dose intravenous administration of 20 mg/kg of the conjugate in HGS to male CD-1 mice (n=4) on the graphs, 2H11-DM4 (bottom), refers to hu2H11R35R74-conjugate. These figures show that for each conjugate, there exists a distribution of conjugates bearing from 0 to 10 maytansinoid(s) (D₀: no maytansinoid; Dₓ: x maytansinoids).

### DETAILED DESCRIPTION

New Maytansinoids

The invention is related to a compound of formula (I):

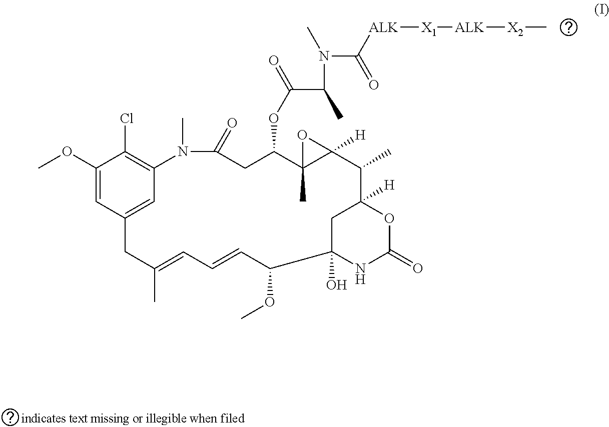

wherein:
ALK is a (C₁-C₆)alkylene group;
X₁ et X₂ are each independently one of the following groups: —CH═CH—, —CO—, —CONR—, —NRCO—, —COO—, —OCO—, —OCONR—, —NRCOO—, —NRCONR′—, —NR—, —S(O)ₙ (n=0, 1 or 2) or —O—;
R and R′ are independently H or a (C₁-C₆)alkyl group;
i is an integer of from 1 to 40, preferably from 1 to 20, and more preferably from 1 to 10;
j is an integer corresponding to 1 when X₂ is —CH═CH— and 2 when X₂ is not —CH═CH—;
Z_{b} is a simple bond, —O— or —NH— and R_{b} is H or a (C₁-C₆)alkyl, (C₃-C₇)cycloalkyl, aryl, heteroaryl or (C₃-C₇)heterocycloalkyl group; or Z_{b} is a single bond and R_{b} is Hal.

More particularly, X₂ is —CH═CH— or —CONR—, the CO group being linked to the —X₁-ALK-group and R being H or a (C₁-C₆)alkyl group. More particularly, —X₁-ALK- is —S—CH₂—. More particularly, i is 3, 4, 5, 6, 7, 8, 9 or 10.

One can distinguish the compound of formula (II):

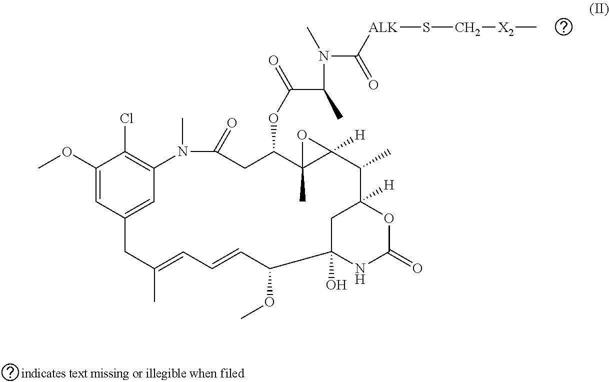

Formula (II) covers more precisely the following compounds:

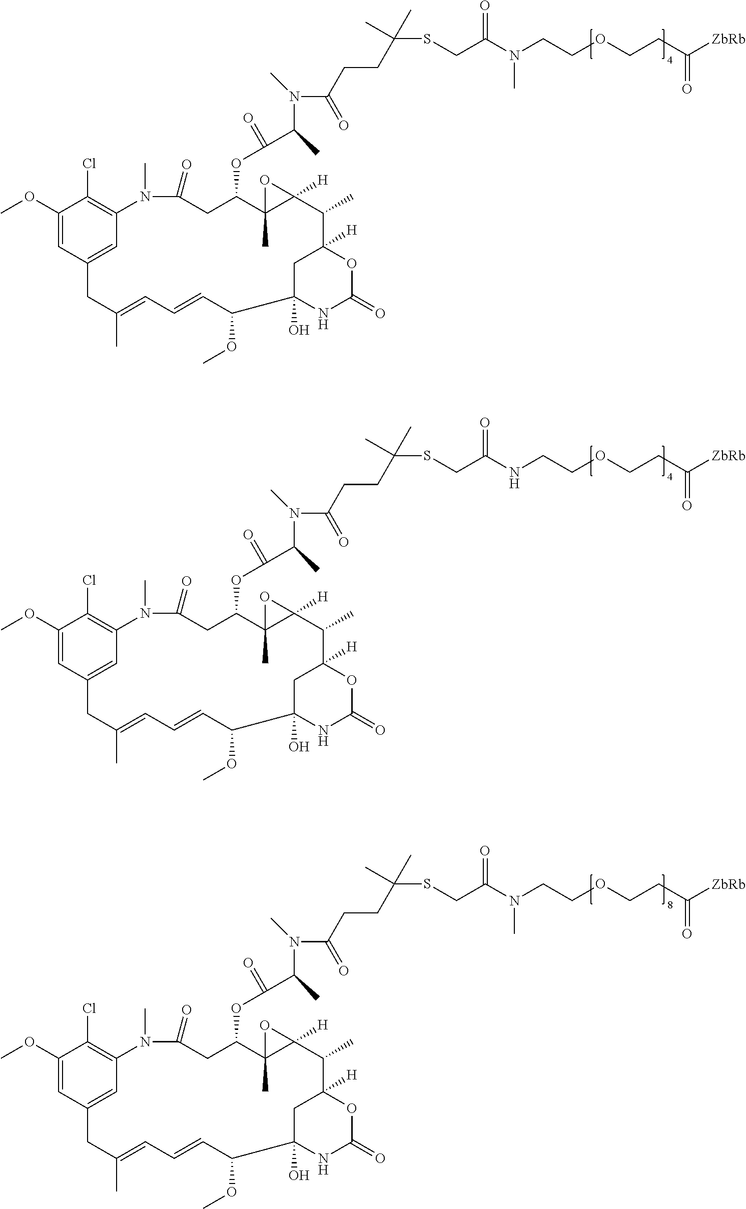

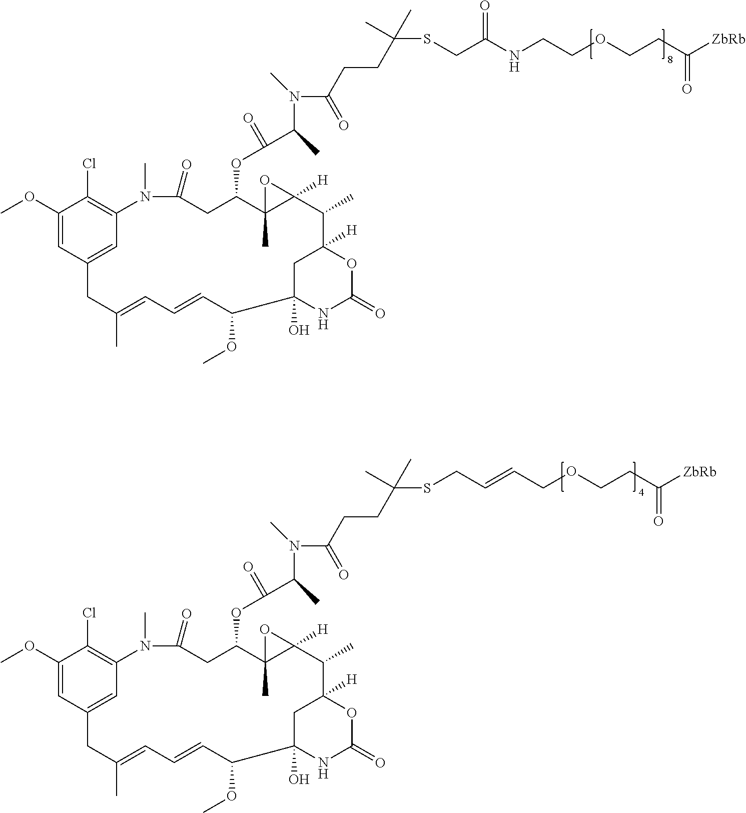

The compounds can exist in the form of a base or a salt or in the form of a solvate or an hydrate of said base or said salt.

The compounds of the invention comprise the reactive chemical group —C(═O)Z_{b}R_{b} (GCR1) which is reactive towards a reactive chemical group (GCR2) that is present on the antibody.

The reaction between GCR1 and GCR2 makes it possible to attach through a covalent bond the cytotoxic agent on the antibody. Thus, the compound is apt to be conjugated to the antibody. More particularly, Z_{b} is O; in such case, GCR1 is an carboxylic acid function (R_{b}=H) or an ester function. More particularly, —C(═O)Z_{b}R_{b} is —COOH, —COO(C₁-C₆)alkyl, like —COOCH₃, or —COOCH₂CH═CH₂. Among the ester functions, the <<activated>> ones that present a good reactivity towards the amino groups of the antibody (like the lysine groups) are preferred. For instance the activated esters may be the following ones:

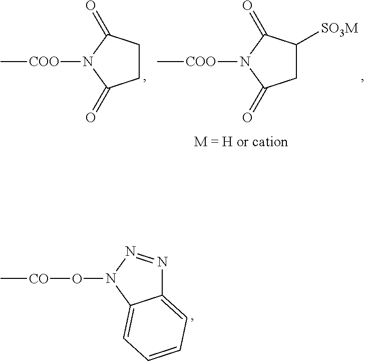

or the group

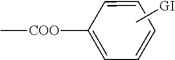

wherein GI represent at least one inductive group like —NO₂ or -Hal, e.g. —F. Examples of such activated esters are the following ones:

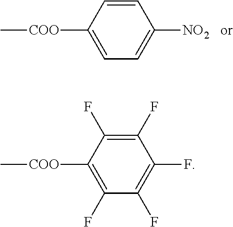

### Another —C(═O)Z_{b}R_{b} is:

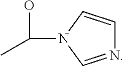

GCR2 may for instance be a ε-amino group born by lysines on the lateral of lysine residues at the surface of an antibody, a saccharide group of the hinging region or the thiol groups of cysteines after reduction of intrachain S—S bonds (Garnett M. C., et al., Advanced Drug Delivery Reviews 2001, 53, 171-216). More recently, new approaches have aimed at introducing cysteines by mutation (Junutula J. R., et al., Nature Biotechnology 2008, 26, 925-932; WO 09026274) or the introduction of non-natural aminoacids making it possible to develop a new type of chemistry of proteins (de Graaf A. J., et al., Bioconjugate Chem. 2009, Feb. 3, 2009 (Review); DOI: 10.1021/bc800294a; WO 2006/069246 and also Chin J. W., et al., JACS 2002, 124, 9026-9027 (technology ReCode®)).

The compounds of the invention can be used to prepare a conjugate on which is covalently attached at least one maytansinoid fragment of formula:

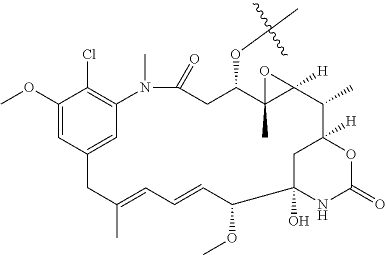

Thus, the compound of formula (I) can be used to prepare a conjugate wherein the maytansinoid fragment is covalently linked to an antibody.

General Scheme to Prepare Compounds of Formula (I)

The compounds of formula (I) can be prepared according to Scheme 1:

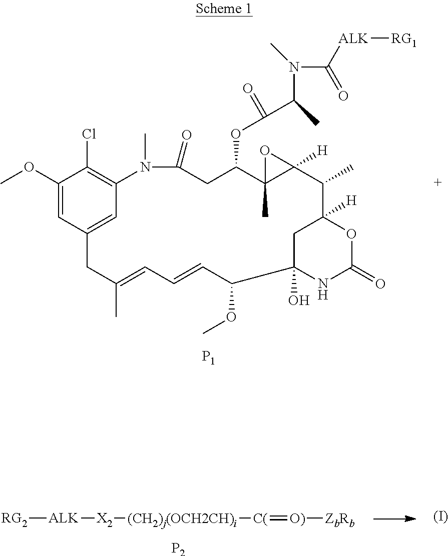

Intermediate P₁ contains a RG₁ reactive group that is able to react with the reactive group RG₂ attached to the PEG containing intermediate P₂ to form X₁. For instance, the formation of X₁=S can be made through the reaction of P₁ with RG₁=—SH and P₂ with RG₂=—Br by a nucleophilic substitution in the presence of a base like DIEA. An example of this reaction is given in ex. 1.2.

Examples of P₁ with RG₁=—SH are L-DM1 and L-DM4 and also the compounds 11a, c, d, g of EP 1313738:
L-DM1: ALK-SH=—CH₂CH₂—SH;
L-DM4: ALK-SH=—CH₂CH₂ CMe₂-SH;
11a: ALK-SH=—CH₂—SH;
11c: ALK-SH=—CH₂CH₂CH₂—SH;
11d: ALK-SH=—CH₂CH₂CH₂CH₂—SH;
11g: ALK-SH=—CHMe-CH₂—SH

Other examples of reactions are given in Table I.

TABLE I

| Entry | X₁ | X₂ | P₂ | RG₁ | RG₂ | conditions of the reaction |
|---|---|---|---|---|---|---|
| 1 | —S— | —CO— | halogeno- | —SH | halogen | nucleophilic substitution of P₁ |
| | | | acetyl | | | with RG₁ = —SH (eg L-DM1 or |
| | | | linker | | | L-DM4) onto an halogeno- |
| | | | | | | acetyl linker in a protic or |
| | | | | | | aprotic polar solvent under |
| | | | | | | neutral or basic conditions |
| | | | | | | (organic, mineral or supported |
| | | | | | | base) |
| 2 | —S— | —CONR— | halogeno- | —SH | halogen | nucleophilic substitution of P₁ |
| | | | amide | | | with RG₁ = —SH (eg L-DM1 or |
| | | | linker | | | L-DM4) onto an halogeno- |
| | | | | | | amide linker in a protic or |
| | | | | | | aprotic polar solvent under |
| | | | | | | neutral or basic conditions |
| | | | | | | (organic, mineral or supported |
| | | | | | | base) |
| 2′ | —S— | —CONR— | sulfonate- | —SH | activated | nucleophilic substitution of P₁ |
| | | | amide | | sulfonate | with RG₁ = —SH (eg L-DM1 or |
| | | | linker | | | L-DM4) onto an activated |
| | | | | | | sulfonate-amide linker (eg |
| | | | | | | mesyl, tosyl, triflate) in a protic |
| | | | | | | or aprotic polar solvent under |
| | | | | | | neutral or basic conditions |
| | | | | | | (organic, mineral or supported |
| | | | | | | base) |
| 3 | —S(O)n- | —CONR— | halogeno- | —SH | halogen | controlled oxidation of Entry 1 |
| | n = 1 | | amide | | | obtained product under mild |
| | or 2 | | linker | | | oxidative conditions (oxone, |
| | | | | | | peracid) in aprotic polar solvant |
| 4 | —S— | —NRCO— | halogeno- | —SH | halogen | nucleophilic substitution of P₁ |
| | | | amide | | | with RG₁ = —SH (eg L-DM1 or |
| | | | linker | | | L-DM4) onto an halogeno- |
| | | | | | | amide linker in a protic or |
| | | | | | | aprotic polar solvent under |
| | | | | | | neutral or basic conditions |
| | | | | | | (organic, mineral or supported |
| | | | | | | base) |
| 5 | —S— | —COO— | halogeno- | —SH | halogen | nucleophilic substitution of P₁ |
| | | | ester | | | with RG₁ = —SH (eg L-DM1 or |
| | | | linker | | | L-DM4) onto an halogeno-ester |
| | | | | | | linker in a protic or aprotic |
| | | | | | | polar solvent under neutral or |
| | | | | | | basic conditions (organic, |
| | | | | | | mineral or supported base) |
| 6 | —S— | —OCO— | halogeno- | —SH | halogen | nucleophilic substitution of P₁ |
| | | | carbonate | | | with RG₁ = —SH (eg L-DM1 or |
| | | | linker | | | L-DM4) onto an halogeno- |
| | | | | | | carbonate linker in a protic or |
| | | | | | | aprotic polar solvent under |
| | | | | | | neutral or basic conditions |
| | | | | | | (organic, mineral or supported |
| | | | | | | base) |
| 7 | —S— | —OCONR— | halogeno- | —SH | halogen | nucleophilic substitution of P₁ |
| | | or | carbamate | | | with RG₁ = —SH (eg L-DM1 or |
| | | —NRCOO— | linker | | | L-DM4) onto an halogeno- |
| | | | | | | carbamate linker in a protic or |
| | | | | | | aprotic polar solvent under |
| | | | | | | neutral or basic conditions |
| | | | | | | (organic, mineral or supported |
| | | | | | | base) |
| 8 | —S— | —RCONR′- | halogeno- | —SH | halogen | nucleophilic substitution of P₁ |
| | | | urea | | | with RG₁ = —SH (eg L-DM1 or |
| | | | linker | | | L-DM4) onto an halogeno-urea |
| | | | | | | linker in a protic or aprotic |
| | | | | | | polar solvent under neutral or |
| | | | | | | basic conditions (organic, |
| | | | | | | mineral or supported base) |
| 9 | —S— | —NR— | halogeno- | —SH | halogen | nucleophilic substitution of P₁ |
| | | | amine | | | with RG₁ = —SH (eg L-DM1 or |
| | | | linker | | | L-DM4) onto an halogeno- |
| | | | | | | amine linker in a protic or |
| | | | | | | aprotic polar solvent under |
| | | | | | | neutral or basic conditions |
| | | | | | | (organic, mineral or supported |
| | | | | | | base) |
| 10 | —S(O)n- | —S(O)n- | halogeno- | —SH | halogen | nucleophilic substitution of P₁ |
| | n = 0, | n = 0, | thio linker | | | with RG₁ = —SH (eg L-DM1 or |
| | 1 or 2 | 1 or 2 | | | | L-DM4) onto an halogeno-thio |
| | | | | | | linker in a protic or aprotic |
| | | | | | | polar solvent under neutral or |
| | | | | | | basic conditions (organic, |
| | | | | | | mineral or supported base) |
| | | | | | | eventually followed by a |
| | | | | | | controlled oxidation of so |
| | | | | | | obtained product under mild |
| | | | | | | oxidative conditions (oxone, |
| | | | | | | peracid) in aprotic polar solvant |
| 11 | —S— | —O— | Halogeno- | —SH | Halogen | nucleophilic substitution of P₁ |
| | | | ether | | | with RG₁ = —SH (eg L-DM1 or |
| | | | linker | | | L-DM4) onto an halogeno- |
| | | | | | | ether linker in a protic or |
| | | | | | | aprotic polar solvent under |
| | | | | | | neutral or basic conditions |
| | | | | | | (organic, mineral or supported |
| | | | | | | base) |

For some compounds of formula (I), the final desired —Z_{b}R_{b} group may be obtained after at least one transformation of another —Z_{b}R_{b} group after reacting P₁ and P₂. An example is given on Scheme 1′ with the transformation —Z_{b}R_{b}=—O-allyle→—Z_{b}R_{b}=

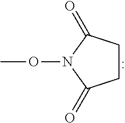

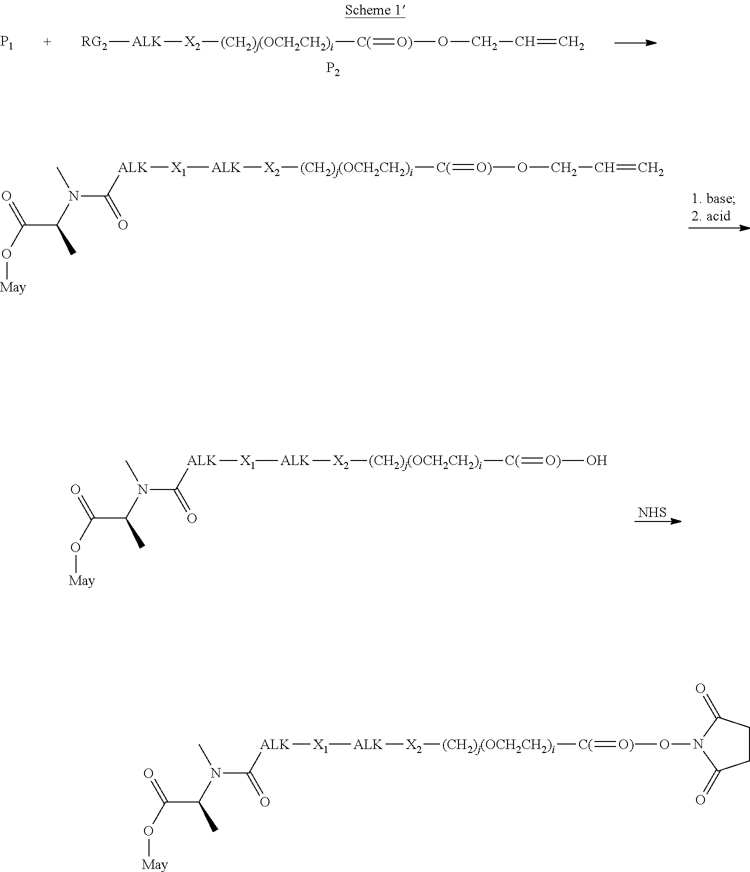

Likewise, at least one transformation may also be used for an intermediate bearing the —Z_{b}R_{b} group, before the reaction between P₁ and P₂.

Another example is the transformation —Z_{b}R_{b}=—OH→—Z_{b}R_{b}=Hal that requires an acylating agent like e.g. SOCl₂.

Preparation of P₁

P₁ can be prepared starting with maytansinol according to Scheme 2:

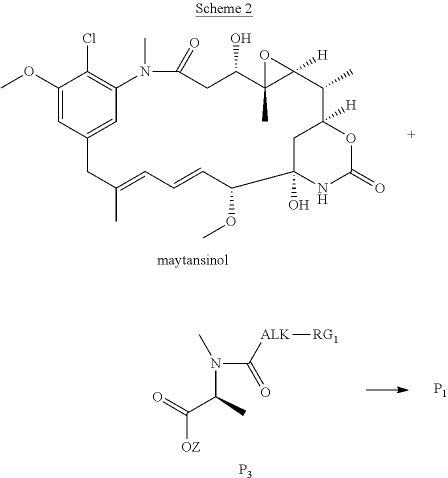

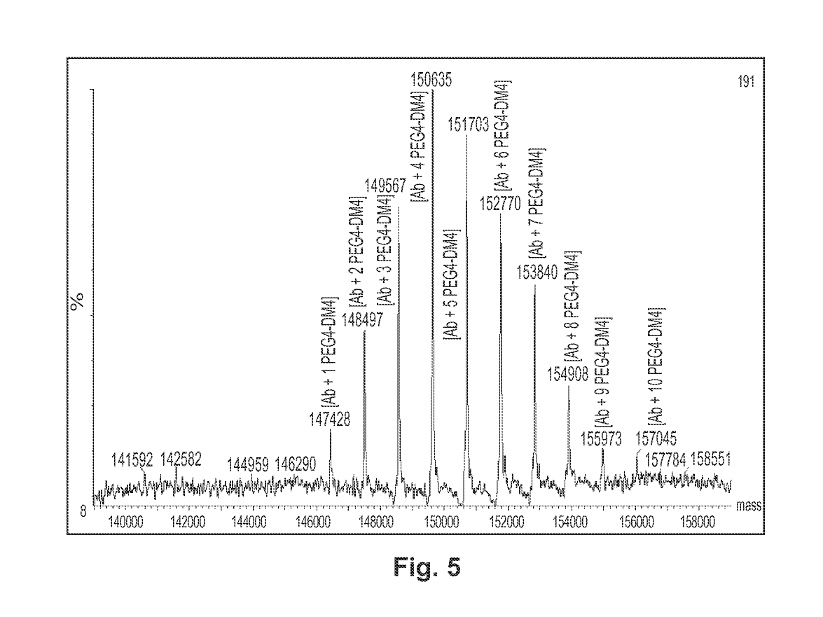

Maytansinol is reacted by an esterification reaction with intermediate P₃ which contains a reactive acyl group —COOZ wherein Z is H or an halogen atom. The reaction is described on FIG. 3a-d of WO 2004/103272 and also in WO 2007/021674. When Z is H, the esterification can be conducted with the aid of a coupling agent that enhances the reactivity of the acid function.

Preparation of P₂

Starting materials to prepare P₂ are PEG compounds that are commercially available or that can be prepared with said commercially available PEG compounds through at least one chemical reaction known to one skilled in the art. PEG compounds are commercially available for instance by JenKem Technology USA Inc. 2033 W. McDermott Dr. Suite 320 #188, Allen, Tex. 75013-4675, USA.

For example, the preparation of P₂ wherein X₂=—CONR— and RG₂=Hal (P₂=Hal-ALK-CONR—CH₂CH₂ (OCH₂CH₂)ᵢ—COZ_{b}R_{b}) is described below with commercially available compounds HOOCCH₂CH₂ (OCH₂CH₂)ᵢ—OCH₂CH₂NH₂:

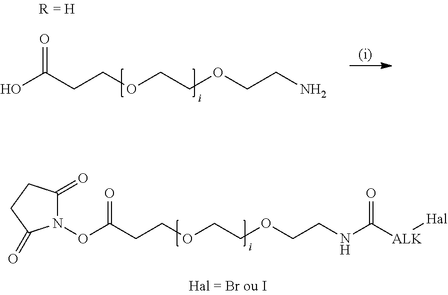

Step (i):

formation of an amide bond and activation of the acid group; the two steps are carried out separately in a polar aprotic solvent like DCM: reaction of the amine group with an halogenoalkanoic acid N-hydroxysuccinimidine ester (eg halogenoacetate) then addition in situ of a coupling agent like DIC.

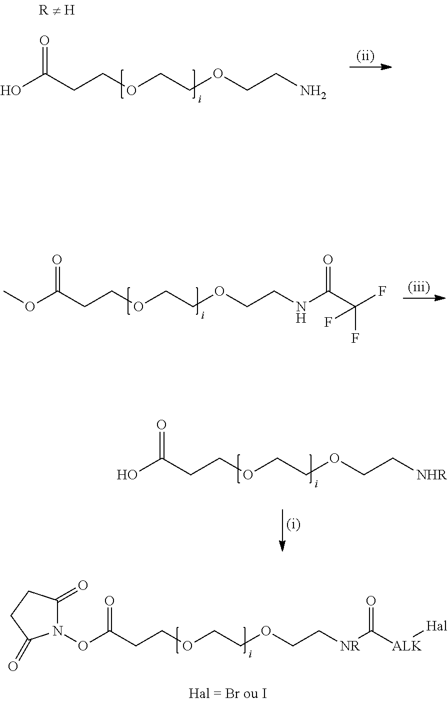

Step (ii):

protection of the carboxylic acid in the form of an ester and of the amine in the form of a trifluoroacetamide; the reaction is carried out in two separate steps in a polar aprotic solvent like DCM: protection of the acid by treatment with trimethylsilyldiazomethane with methanol then protection of the amine by addition of TFAA and TEA;

Step (iii):

alkylation of the amine and alkaline hydrolysis of the ester: the reaction is carried out in two separate steps in a polar aprotic solvent like THF:alkylation of the amine by treatment with NaH in the presence of a reactant bearing a leaving group like an alkyl halide RHal, and addition of LiOH in water;

Step (i):

following step (iii), the reactions of step (i) where R=H are carried out.

Likewise, the preparation of P₂ wherein X₂=—CH═CH— and RG₂=Hal (P₂=Hal-CH₂—CH═CH—CH₂(OCH₂CH₂); —COZ_{b}R_{b}) is described below with commercially available compounds Hal-CH₂ CH═CH₂CH₂ (OCH₂C₂CH)—COOtBu:

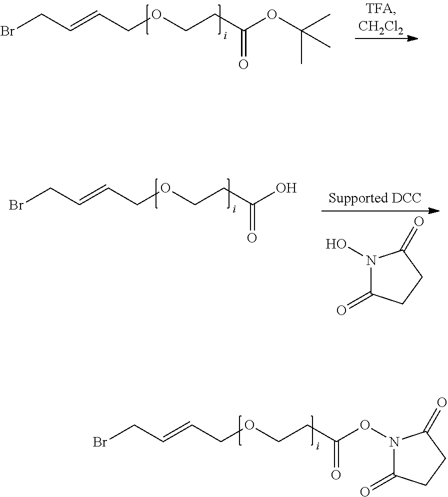

Process of Preparation of the Conjugates

The conjugate can be obtained by a process comprising the steps of:

(i) bringing into contact an optionally-buffered aqueous solution of the antibody with a solution of a compound of formula (I):

(ii) then optionally separating the conjugate which was formed in (i) from the unreacted reagents and any aggregate which may be present in the solution.

The aqueous solution of cell-binding agent can be buffered with at least one buffer such as, e.g. potassium phosphate or N-2-Hydroxyethylpiperazine-N′-2-ethanesulfonic acid (Hepes buffer) or a mixture of buffers such as e.g. buffer A disclosed in the examples below. The buffer depends upon the nature of the antibody. The compound of formula (I) is in solution in an organic polar solvent (or a mixture of polar solvents), e.g. DMSO or DMA.

The temperature of the reaction usually varies from 20 to 40° C. The reaction time can vary from 1 to 24 hours. The reaction between the antibody and the cytotoxic agent can be monitored by size exclusion chromatography (SEC) with a refractometric and/or UV detector. If the conjugate yield is too low, the reaction time can be extended and/or the compound of formula (I) can be added.

A number of different chromatography methods can be used by the person skilled in the art in order to perform the separation of step (ii): the conjugate can be purified e.g. by SEC, adsorption chromatography (such as ion exchange chromatography, IEC), hydrophobic interaction chromatography (HIC), affinity chromatography, mixed-support chromatography such as hydroxyapatite chromatography, or high performance liquid chromatography (HPLC). Purification by dialysis or diafiltration can also be used.

An example of a process which can be used is described in the Example I.

As used herein, the term “aggregates” means the associations which can be formed between two or more antibodies, said antibodies being modified or not by conjugation. The aggregates can be formed under the influence of a great number of parameters, such as a high concentration of antibody in the solution, the pH of the solution, high shearing forces, the number of bonded dimers and their hydrophobic character, the temperature (see Wang & Gosh, 2008, Membrane Sci., 318: 311-316, and references cited therein); note that the relative influence of some of these parameters is not clearly established. In the case of proteins and antibodies, the person skilled in the art will refer to Cromwell et al. (2006, AAPS Journal, 8(3): E572-E579). The content in aggregates can be determined with techniques well known to the skilled person, such as SEC (see Walter et al., 1993, Anal. Biochem., 212(2): 469-480).

After step (i) or (ii), the conjugate-containing solution can be submitted to an additional step (iii) of ultrafiltration and/or diafiltration.

The conjugate is recovered at the end of these steps as an aqueous solution.

Antibody

The term “antibody” is used herein in the broadest sense and specifically covers monoclonal antibodies (including full length monoclonal antibodies) of any isotype such as IgG, IgM, IgA, IgD, and IgE, polyclonal antibodies, multispecific antibodies, chimeric antibodies, and antibody fragments. An antibody reactive with a specific antigen can be generated by recombinant methods such as selection of libraries of recombinant antibodies in phage or similar vectors, or by immunizing an animal with the antigen or an antigen-encoding nucleic acid.

A typical antibody is comprised of two identical heavy chains and two identical light chains that are joined by disulfide bonds. Each heavy and light chain contains a constant region and a variable region. As used herein, “V_{H}” or “VH” refers to the variable region of an immunoglobulin heavy chain of an antibody, including the heavy chain of an Fv, scFv, dsFv, Fab, Fab′, or F(ab′)2 fragment. Reference to “V_{L}” or “VL” refers to the variable region of the immunoglobulin light chain of an antibody, including the light chain of an Fv, scFv, dsFv. Fab, Fab′, or F(ab′)2 fragment. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions are called the “framework regions” (“FR”). The variable domains of native heavy and light chains each comprise four FR regions, broadly adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, 5^{th} edition, National Institute of Health, Bethesda, Md., 1991).

The antibody (see for more details, Janeway et al. <<Immunobiology>>, 5^{th} ed, 2001, Garland Publishing, New York) can be chosen for instance among those mentioned in WO 04043344, WO 08010101, WO 08047242 or WO 05009369 (anti-CA6).

The antibody or fragments thereof that recognize class A Eph receptor family members, such as EphA2 receptor, preferably human, and function as antagonists of said receptor, can also be considered. This antibody is devoid of any agonist activity. The antibody or an epitope-binding fragment thereof can be one described in claims 12-15.

The humanized antibody or epitope-binding fragments thereof preferably have the additional ability to inhibit growth of a cancer cell expressing the EphA2 receptor. The humanized antibody or epitope-binding fragment thereof has preferably the additional ability to inhibit the migration of a metastatic cancer cell expressing the EphA2 receptor.

The humanized antibody can be a humanized 2H11R35R74 antibody, or an epitope-binding fragment thereof. An humanized antibody can be obtained by site-directed mutagenesis of the polynucleotide sequences encoding hu53.2H11 (WO 2008/010101). Preferably, there are provided resurfaced or humanized versions of the 2H11R35R74 antibody wherein surface-exposed residues of the antibody or its fragments are replaced in both light and heavy chains to more closely resemble known human antibody surfaces. The humanized 2H11R35R74 antibody or epitope-binding fragments thereof have improved properties. For example, humanized 2H11R35R74 antibodies or epitope-binding fragments thereof specifically recognize EphA2 receptor. More preferably, the humanized 2H11R35R74 antibody or epitope-binding fragments thereof have the additional ability to inhibit the growth of an EphA2 receptor-expressing cell.

The humanized versions of the 2H11R35R74 antibody are also fully characterized herein with respect to their respective amino acid sequences of both light and heavy chain variable regions, the DNA sequences of the genes for the light and heavy chain variable regions, the identification of the CDRs, the identification of their surface amino acids, and disclosure of a means for their expression in recombinant form. However, the scope is not limited to antibodies and fragments comprising these sequences. Instead, all antibodies and fragments that specifically bind to EphA2 receptor are also considered. Preferably, the antibodies and fragments that specifically bind to EphA2 receptor antagonize the biological activity of the receptor. More preferably, such antibodies further are substantially devoid of agonist activity. Thus, antibodies and epitope-binding antibody fragments may differ from the 2H11R35R74 antibody or the humanized derivatives thereof, in the amino acid sequences of their scaffold, CDRs, and/or light chain and heavy chain, and still fall within the scope of the present invention.

The CDRs of the 2H11R35R74 antibody are identified by modeling and their molecular structures have been predicted. Again, while the CDRs are important for epitope recognition, they are not essential to the antibodies and fragments of the invention. Accordingly, antibodies and fragments are provided that have improved properties produced by, for example, affinity maturation of an antibody of the present invention.

The mouse light chain IgVκ and Jκ germline genes and heavy chain IgVh and Jh germline genes from which 53.2H11 was likely derived have been identified, and were disclosed in WO 2008/010101. The accession numbers of said germline sequences are respectively MMU231196 and AF303833. Such germline gene sequences are useful to identify somatic mutations in the antibodies, including in the CDRs.

The sequences of the heavy chain and light chain variable regions of the 2H11R35R74 antibody, and the sequences of their CDRs are set forth in this application. Such information can be used to produce humanized versions of the 2H11R35R74 antibody. It is also possible to obtain the humanized 2H11R35R74 antibodies of the invention by site-directed mutagenesis of hu53.2H11. These humanized anti-EphA2 antibodies or their derivatives may also be used as the cell binding agent of the conjugates of the present invention.

Thus, in one embodiment, this invention provides humanized antibodies or epitope-binding fragment thereof comprising one or more CDRs having an amino acid sequence selected from the group consisting of SEQ ID NOS: 1, 2, 3, 4, 5, 6. In a preferred embodiment, the humanized antibodies of the invention comprise at least one heavy chain and at least one light chain, wherein said heavy chain comprises three sequential CDRs having amino acid sequences represented by SEQ ID NOS: 1, 2, and 3, and wherein said light chain comprises three sequential CDRs having amino acid sequences represented by SEQ ID NOS: 4, 5, and 6.

The humanized 2H11R35R74 antibody or fragments thereof preferably comprises a V_{H} having an amino acid sequence consisting of SEQ ID NO. 12. A humanized 2H11R35R74 antibody or fragments thereof which comprises a V₁ having an amino acid sequence consisting of SEQ ID NO 14 is also preferred. Preferably the humanized 2H11R35R74 antibody comprises at least one heavy chain and at least one light chain, wherein said heavy chain comprises three sequential CDRs having amino acid sequences represented by SEQ ID NOS: 1, 2, and 3, wherein said light chain comprises three sequential CDRs having amino acid sequences represented by SEQ ID NOS: 4, 5, and 6, wherein said heavy chain has an amino acid sequence consisting of SEQ ID NO. 12, and wherein said light chain has an amino acid sequence consisting of SEQ ID NO. 14.

Conjugate

A conjugate comprises generally from 1 to 10 molecule(s) of the maytansinoid attached covalently to the antibody (so called, “drug-to-antibody ratio” or “DAR”). This number can vary with the nature of the antibody and of the maytansinoid used along with the experimental conditions used for the conjugation (like the ratio maytansinoid/antibody, the reaction time, the nature of the solvent and of the cosolvent if any). Thus the contact between the antibody and the maytansinoid leads to a mixture comprising: several conjugates differing from one another by different drug-to-antibody ratios:optionally the naked antibody:optionally aggregates. The DAR that is determined is thus a mean value.

The invention is therefore also related to a conjugate comprising one or more compound(s) as defined in one of claims 1-8 covalently attached to an antibody. The attachment is preferably through an amide bond. The antibody is preferably as defined in any one of claims 12-15.

The method used herein to determine the DAR consists in measuring spectrophotometrically the ratio of the absorbance at 252 nm and 280 nm of a solution of the substantially purified conjugate (that is after step (ii)). In particular, said DAR can be determined spectrophotometrically using the measured extinction coefficients at respectively 280 and 252 nm for the antibody: ε_{A280}=224,000 M⁻¹cm⁻¹ and ε_{A252}=82,880 M⁻¹cm⁻¹; assuming an average 160,000 molecular weight for the antibody, and for the maytansinoid, ε_{D280}=5,180 M⁻¹cm⁻¹ and ε_{D252}=26,159 M⁻¹cm⁻¹). The method of calculation is derived from Antony S. Dimitrov (ed), LLC, 2009, Therapeutic Antibodies and Protocols, vol 525, 445, Springer Science and is described in more details below:

The absorbances for the conjugate at 252 nm (A₂₅₂) and at 280 nm (A₂₈₀) are measured either on the monomeric peak of the SEC analysis (allowing to calculate the “DAR(SEC)” parameter) or using a classic spectrophotometer apparatus (allowing to calculate the “DAR(UV)” parameter). The absorbances can be expressed as follows:

A₂₅₂=(c_{D}×ε_{D252})+(c_{A}×ε_{A252})

A₂₈₀=(c_{D}×ε_{D280})+(c_{A}×ε_{A280})

wherein:
c_{D} and c_{A} are respectively the concentrations in the solution of the maytansinoid and of the antibody
ε_{D252} and ε_{D280} are respectively the molar extinction coefficients of the maytansinoid at 252 nm and 280 nm
ε_{A252} and ε_{A280} are respectively the molar extinction coefficients of the antibody at 252 nm and 280 nm.

Resolution of these two equations with two unknowns leads to the following equations:

c_{D}=[(ε_{A280}×A₂₅₂)−(ε_{A252}×A₂₈₀)]/[(ε_{D252}×ε_{A280})−(ε_{A252}×ε_{D280})]

c_{A}=[A₂₈₀−(c_{D}×ε_{D280})]/ε_{A280}

The average DAR is then calculated from the ratio of the drug concentration to that of the antibody:

DAR=c_{D}/c_{A}

The average DAR measured with a UV spectrophotometer (DAR(UV)) is more particularly above 4, more particularly between 4 and 10, even more particularly between 4 and 7.

The conjugate and also the compound of formula (I) can be used as anticancer agents. Advantageously, the antibody makes it possible to have an agent that is selective towards the tumour cells, thus targeting the maytansinoid to a close vicinity of said cells or directly within them (see <<Antibody-drug conjugates for cancer therapy>> Carter P. J. et al., Cancer J. 2008, 14, 154-169: <<Targeted cancer therapy: conferring specificity to cytotoxic drugs>> Chari R., Acc. Chem. Res. 2008, 41, 98-107). Solid or liquid tumours can be treated.

The conjugate can be formulated in the form of an aqueous buffered solution, preferably at a concentration between 1 and 10 mg/ml. The solution can be administered as such or it can be diluted to form a solution for perfusion.

### EXAMPLES

### Method A

### High Pressure Liquid Chromatography-Mass Spectrometry (LCMS)

Spectra have been obtained on a Waters UPLC-SQD system in positive and/or negative electrospray mode (ES+/−). Chromatographic conditions are the following: column: ACQUITY BEH C18, 1.7 μm-2.1×30 mm; solvents: A: H₂O (0.1% formic acid) B: CH₃CN (0.1% formic acid); column temperature: 45° C.; flow rate; 0.6 ml/min; gradient (2 min): from 5 to 50% of B in 1 min; 1.3 min: 100% of B; 1.45 min: 100% of B; 1.75 min: 5% of B.

### Method B

### High Pressure Liquid Chromatography-Mass Spectrometry (LCMS)

Spectra have been obtained on a Waters ZQ system in positive and/or negative electrospray mode (ES+/−). Chromatographic conditions are the following: column: XBridge C18 2.5 μm 3×50 mm; solvents: A: H₂O (0.1% formic acid) B: CH₃CN (0.1% formic acid; column temperature: 70° C.; flow rate: 0.9 ml/min; gradient (7 min): from 5 to 100% of B in 5.3 min; 5.5 min: 100% of B; 6.3 min: 5% of B.

### Method C

### Mass Spectrometry (MS)

Spectra have been obtained on a Waters HPLC-SQD system in positive and/or negative electrospray mode (ES+/−). Chromatographic conditions are the following: column: ACQUITY BEH C18 1.7 μm-2.1×50 mm; solvents: A: H₂O (0.1% formic acid) B: CH₃CN (0.1% formic acid); column temperature: 50° C.; flow rate: 1 ml/min; gradient (2 min): from 5 to 50% of B in 0.8 min; 1.2 min: 100% of B; 1.85 min: 100% of B; 1.95: 5% of B.

### Method D

### High Pressure Liquid Chromatography-Mass Spectrometry (LCMS)

Spectra have been obtained on a Waters HPLC-SQD system in positive and/or negative electrospray mode (ES+/−). Chromatographic conditions are the following: column: ACQUITY BEH C18 1.7 μm-2.1×50 mm; solvents: A: H₂O (0.1% formic acid) B: CH₃CN (0.1% formic acid); column temperature: 50° C.; flow rate: 1 ml/min; gradient (2 min): from 5 to 50% of B in 0.8 min; 1.2 min: 100% of B; 1.85 min: 100% of B; 1.95: 5% of B.

### Method G

### Deglycosylation and High Resolution Mass Spectrometry of Conjugates (HRMS)

Deglycosylation is a technique of enzymatic digestion by means of glycosidase. The deglycosylation is made from 500 μl of conjugated+100 μl of Tris buffer HCl 50 mM+10 μl of glycanase-F enzyme (100 units of freeze-dried enzyme/100 μl of water). The medium is vortexed and maintained one night at 37° C. The deglycosylated sample is then ready to be analyzed in HRMS. Mass spectra were obtained on a Waters Q-T of-2 system in electrospray positive mode (ES+). Chromatographic conditions are the following: column: 4 μm BioSuite 250 URH SEC 4.6×300 mm (Waters); solvents: A: ammonium formate 25 mM+1% formic acid: B: CH₂CN; column temperature: 30° C.; flow rate 0.4 ml/min; isocratic elution 70% A+30% B (15 min).

### Method H

### Analytical Size Exclusion Chromatography (SEC)

Column: TSKgel G3000 SWXL 5 μm column, 7.8 mm×30 cm, TOSOH BIOSCIENCE, LLC Part #08541
Mobile Phase: KCl (0.2 M), KH₂PO₄ (0.052 M), K₂HPO₄ (0.107 M), iPrOH (20% in volume)
Analysis Conditions: isocratic elution at 0.5 ml/min for 30 min
Analysis performed on a Lachrom Elite HPLC system (Merck) using a L2455 DAD spectrophotometer detector.

Buffers Contents
Buffer A (pH 6.5): NaCl (50 mM), Potassium Phosphate buffer (50 mM), EDTA (2 mM)
Buffer HGS (pH 5.5): histidine (10 mM), glycine (130 mM), sucrose 5% (w/v), HCl (8 mM)

Abbreviations Used

AcOEt: ethyl acetate; ALK: (C₁-C₁₂)alkylene group, particularly (C₁-C₆)alkylene; DAR: Drug Antibody Ratio; DBU: 1,8-diazabicyclo[5.4.0]undec-7-ene; DCC: N,N′-dicyclohexylcarbodiimide; DCM: dichloromethane; DEAD: diethylazodicarboxylate; DIC: N,N′-diisopropylcarbodiimide; DIPEA: N,N-diisopropylethylamine; DMA: dimethylacetamide; DMAP: 4-dimethylaminopyridine; DME: dimethoxyethane; DMF: dimethylformamide; DMSO: dimethylsulfoxyde; □: molar extinction coefficient; EEDQ: 2-ethoxy-1-ethoxycarbonyl-1,2-dihydroquinoline; EDCl: N-(3-dimethylaminopropyl)-N′-ethylcarbodiimide; EDTA: ethylene-diamine-tetraacetic acid; Fmoc: fluorenylmethoxycarbonyl; Hal: halogen atom; HOBt: 1-hydroxybenzotriazole; HEPES: 4-(2-hydroxyethyl)-1-piperazine-ethanesulfonic acid; HRMS: High Resolution Mass Spectroscopy; NHS: N-hydroxysuccinimide; iPrOH: iso-propyl alcohol; NMP: N-methylpyrrolidinone; Rf: retention factor; RP: reduced pressure; RT: room temperature; SEC: Size Exclusion Chromatography; TBDMS: tert-butyldimethylsilyl; TEA: triethylamine; TFA: trifluoroacetic acid; TFAA: trifluoroacetic anhydride; TFF: Tangential Flow Filtration; THF: tetrahydrofurane; TIPS: triisopropylsilyl; TLC: Thin Layer Chromatography; t_{R}: retention time.

Antibodies Used in the Examples

Two Antibodies were Used to Prepare the Conjugates:
hu2H11: (also referenced hu53 2H11 in WO 2008010101): the antibody is produced by a hybridoma deposited under the Budapest Treaty at the American Type Culture Collection, under the accession number PTA-7662, and is described in PCT application WO 2008/010101 ;
hu2H11R35R74: this humanized antibody binds to an EphA2 receptor and is obtained by site-directed mutagenesis of hu53 2H11, consisting of heavy chain of sequence SEQ ID NO: 18 and light chain of SEQ NO: 16.

### Example I

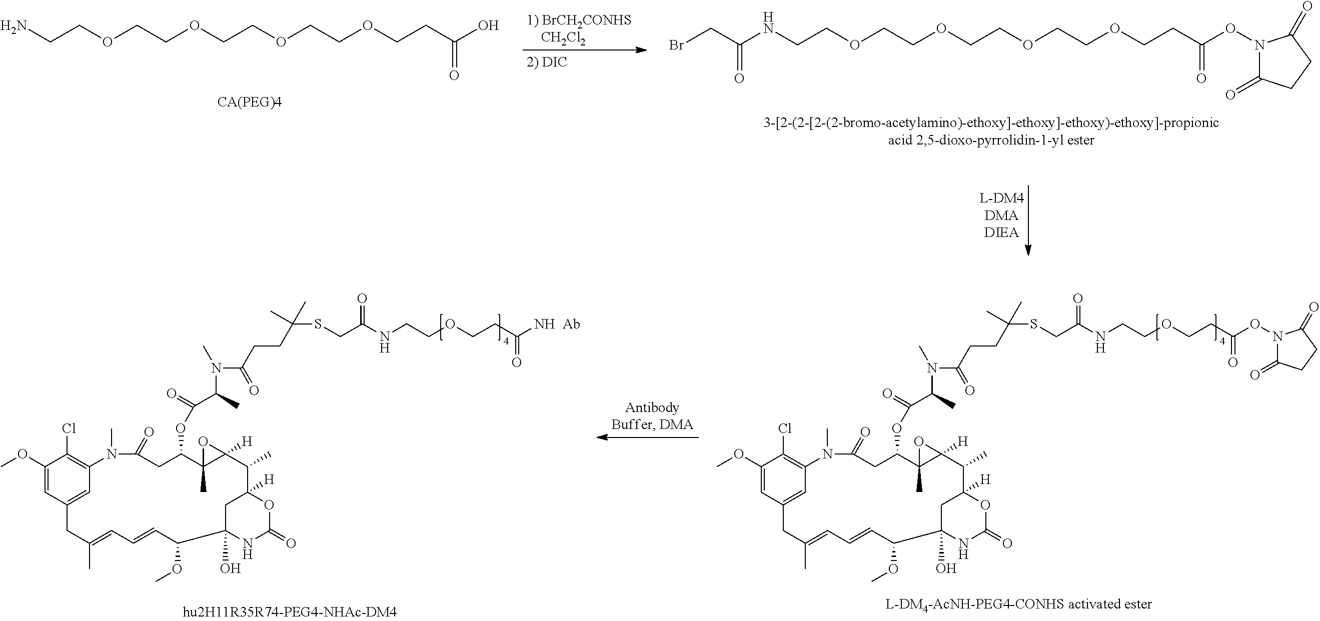

### 1.1. Preparation of Conjugate hu2H11R35R74-PEG4-NHAc-DM4

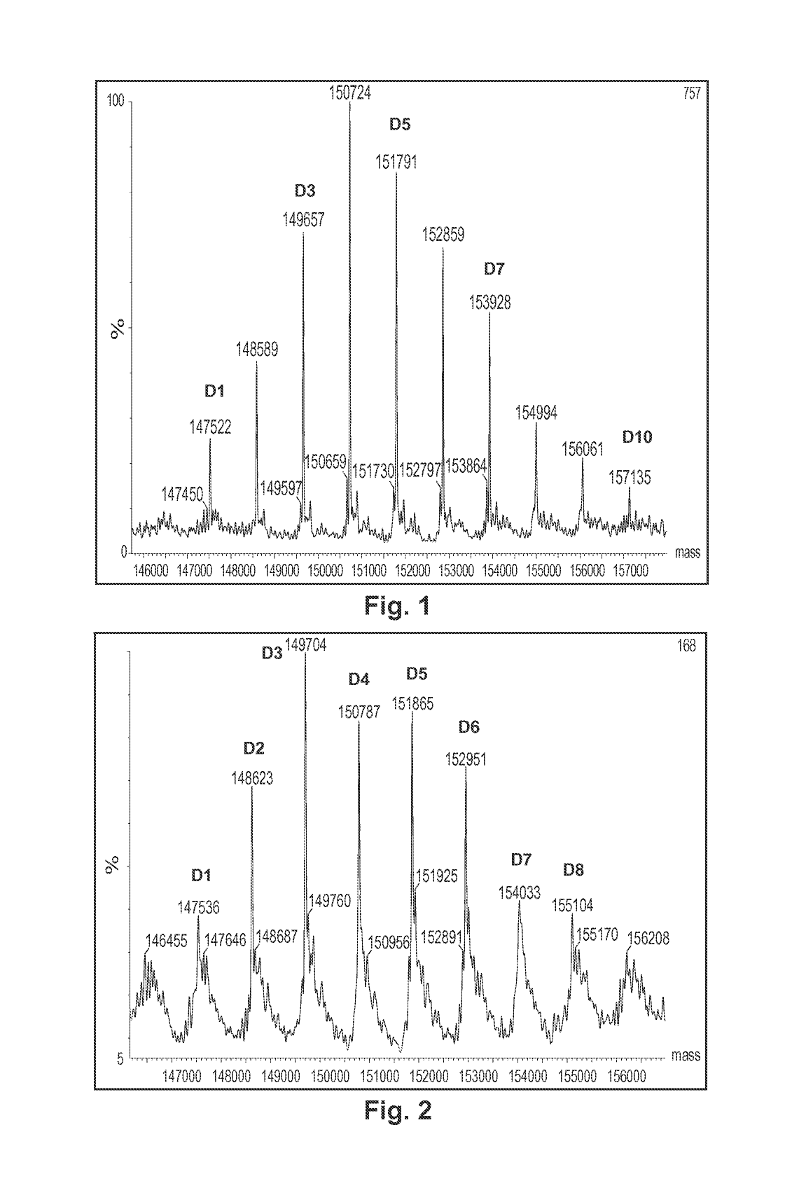

Under magnetic stirring, at RT, 9 ml of hu2H11R35R74 (14.36 mg/ml in buffer A) are added, then 16.85 ml of buffer A, 3.23 ml of HEPES 1M, 1.59 ml of DMA, followed by 1.64 ml of a mM DMA solution of L-DM4-AcNH-PEG4-CONHS activated ester. After 1 hr 30 min at RT, an extra 0.085 ml of 10 mM DMA solution of L-DM4-AcNH-PEG4-CONHS activated ester is added. After 1 hours 45 min at RT, the crude conjugation medium is diluted with 60 ml of HGS buffer and purified by TFF on Pellicon 3 cassettes. The sample is diafiltered against ˜10 sample volumes of HGS buffer and then collected. The TFF tank and lines are washed with an extra 10 ml of HGS buffer. The two solutions are mixed, filter-sterilized through 0.22 μm PVDF, concentrated on Amicon 15 and filter-sterilized through 0.22□m PVDF. 17 ml of hu2H11R35R74-PEG4-NHAc-DM4 conjugate (c=5.76 mg/ml) was thus obtained. The conjugate is then analyzed for final drug load and monomeric purity. SEC analysis (method H): DAR (SEC)=5.4; RT=16.757 min; monomeric purity=99.5%; HRMS data: see FIG. 1.

### 1.2. Preparation of L-DM4-AcNH-PEG4-CONHS Activated Ester

Under magnetic stirring at RT, 154.3 mg of L-DM4 (prepared according to WO 04/103272—see compounds 4b) are introduced in a glass vial. A solution of 90 mg of 3-[2-(2-{2-[2-(2-bromo-acetylamino)-ethoxy]-ethoxy}-ethoxy)-ethoxy]-propionic acid 2,5-dioxo-pyrrolidin-1-yl ester in 0.94 ml of DMA is then added, followed by 36 μl of DIEA. After 23 hrs at RT, the reaction medium is diluted with 5 ml of AcOEt and washed with 7 ml of water. The aqueous phase is extracted with 5 ml of AcOEt. The combined organic phases are dried over MgSO₄, concentrated to dryness under RP. 228 mg of pale yellow viscous oil are obtained, which product is diluted with a minimum amount of DMA and purified by flash-chromatography on 30 g of C18-grafted silica gel (gradient of elution water:acetonitrile 95:5 to 5:95 by volume). After concentration of fractions 2 and 3 under RP, a colourless viscous oil is obtained, which product is diluted with a minimum amount of DMA and purified by flash-chromatography on 30 g of C18-grafted silica gel (gradient of elution water:acetonitrile 95:5 to 5:95 by volume). After concentration of fractions 33 to 35 under RP, 41 mg of L-DM4-AcNH-PEG4-CONHS activated ester are obtained in the form of a white meringue-like product. Mass spectra (B): RT=4.06 min; [M+H−H₂O]+: m/z 1164; [M+H]+: m/z 1182; [M−H+HCO₂H]−: m/z 1226; ¹H NMR (500 MHz, δ in ppm, chloroform-d): 0.80 (s, 3H); 1.21 (s, 3H); 1.22 (s, 3H); 1.25 (m, 1H); 1.29 (d, J=6.7 Hz, 6H); 1.46 (m, 1H); 1.57 (d, J=13.4 Hz, 1H); 1.64 (s, 3H); 1.76 to 1.83 (m, 1H); 1.88 to 1.96 (m, 1H); 2.18 (dd, J=2.5 et 14.3 Hz, 1H); 2.36 (m, 1H); 2.53 (m, 1H); 2.61 (dd, J=12.5 and 14.3 Hz, 1H); 2.82 to 2.92 (m, 10H; 2.98 (d, J=16.7 Hz, 1H); 3.03 (d, J=9.6 Hz, 1H); 3.15 (d, J=12.9 Hz, 1H); 3.22 (s, 3H); 3.32 (s broad, 1H); 3.36 (s, 3H); 3.42 (m, 2H); 3v50 (d, J=9.1 Hz, 1H); 3.53 (t, J=5.2 Hz, 2H); 3.58 to 3v67 (m, 13H); 3.84 (t, J=6.4 Hz, 2-H); 3.99 (s, 3H); 4.27 (m, 1H); 4.77 (dd, J=2.9 and 11.9 Hz, 1H); 5.42 (q, J=6.7 Hz, 1H); 5.66 (dd, J=9.1 and 15.4 Hz, 1H); 6.23 (s, 1H); 6.43 (dd, J=11.3 and 15.4 Hz, 1H); 6.64 (d, J=1.1 Hz, 1H); 6.74 (d, J=11.3 Hz, 1H); 6.85 (d, J=1.1 Hz, 1H); 7.08 (t, J=5.2 Hz, 1H)

### 1.3. Preparation of 3-[2-(2-{2-[2-(2-bromo-acetylamino)-ethoxy]-ethoxy}-ethoxy)-ethoxy]-propionic acid 2,5-dioxo-pyrrolidin-1-yl ester

Under magnetic stirring at RT, 671.4 mg of 3-(2-{2-[2-(2-amino-ethoxy)-ethoxy]-ethoxy}-ethoxy)-propionic acid (CA(PEG)₄, Pierce) are introduced in a glass vial. A solution of 597.4 mg of bromo-acetic acid 2,5-dioxo-pyrrolidin-1-yl ester in 14 ml of DCM is then added. After min at RT, 0.396 ml of DIC is added. After 1 hr 30 min, the crude reaction medium is filtered on sintered glass, and the filtrate is purified by flash-chromatography on 100 g of CN-grafted silica gel (gradient of elution n heptane/iPrOH/AcOEt with increasing iPrOH portion). After concentration of fractions 30 to 45 under RP, 761 mg of 3-[2-(2-{2-[2-(2-bromo-acetylamino)-ethoxy]-ethoxy}-ethoxy)-ethoxy]-propionic acid 2,5-dioxo-pyrrolidin-1-yl ester are obtained in the form of a colourless oil. Mass spectra (A); RT=0.74 min; [M+H]+: m/z 483/485 (two peaks due to the two isotopes of Br); [M−H+HCO₂H]−: m/z 527/529 (two peaks due to the two isotopes of Br).

Bromo-acetic acid 2,5-dioxo-pyrrolidin-1-yl ester could be prepared following published protocol (Biochemistry 1974, 481).

### Example 2

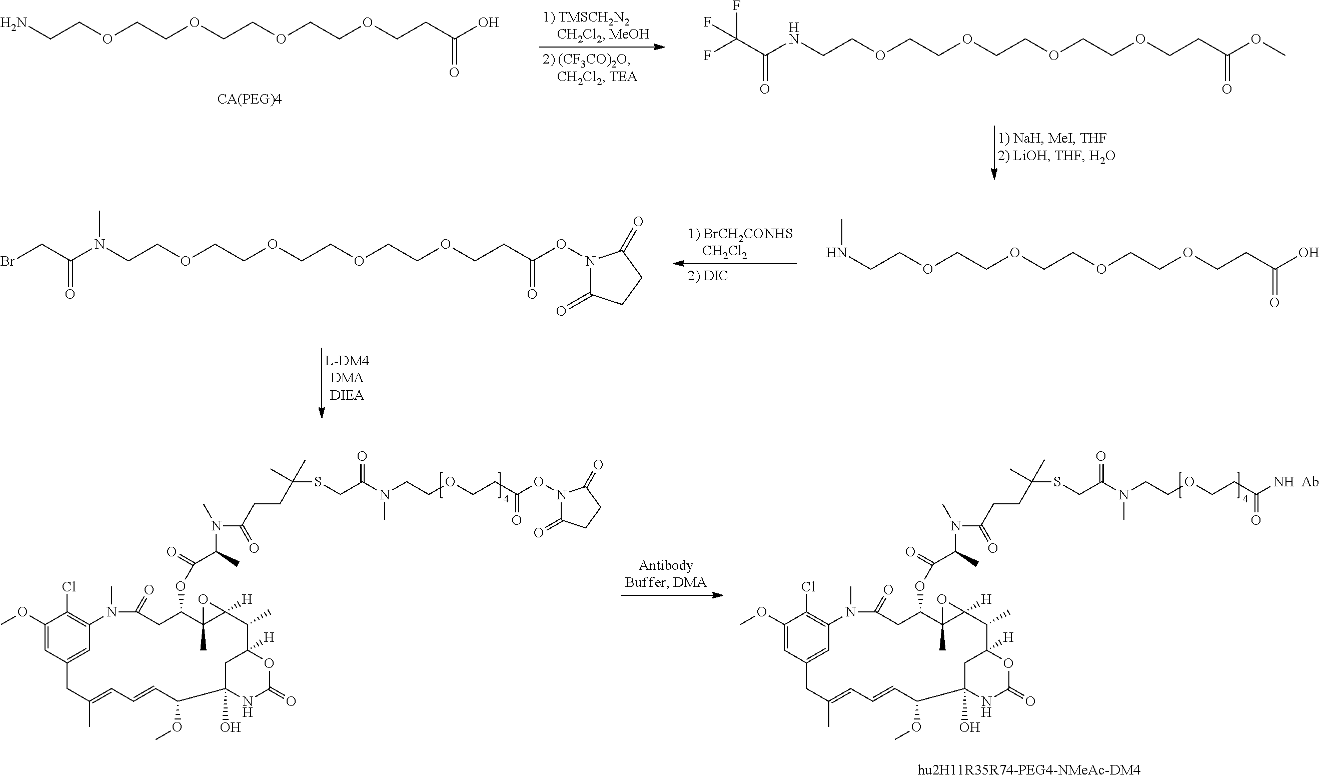

### 2.1. Preparation of Conjugate hu2H11R35R74-PEG4-NMcAc-DM 4

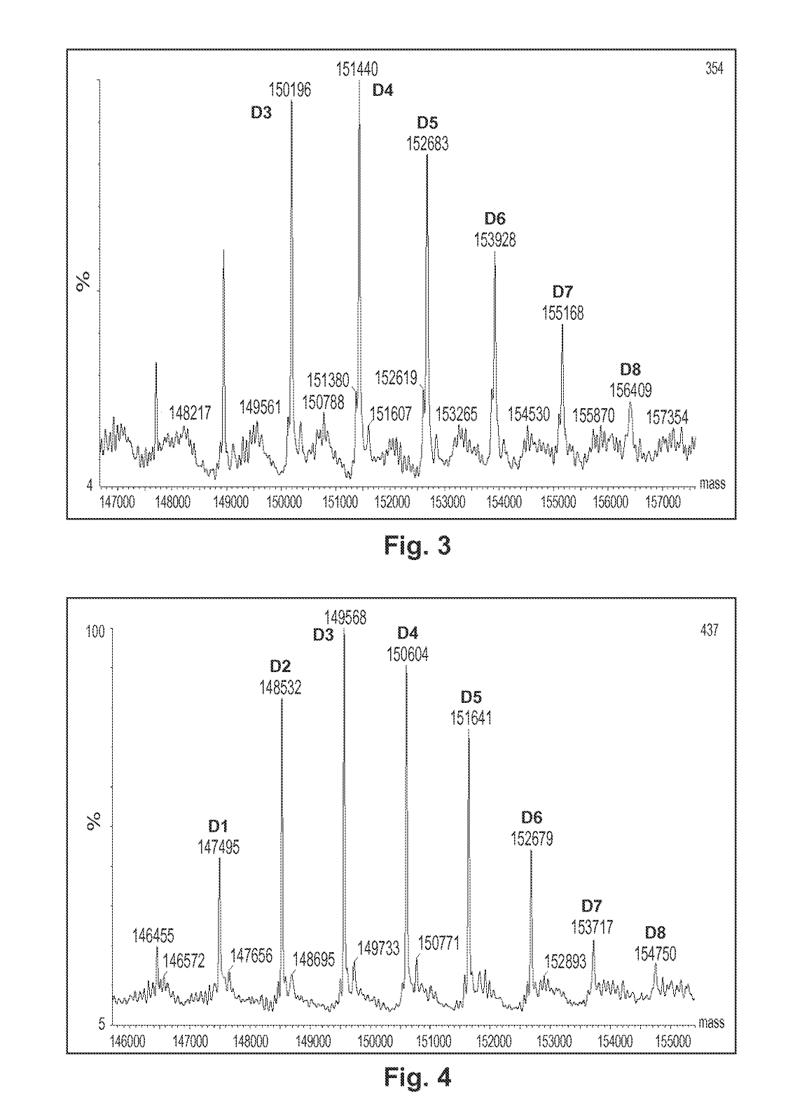

Under magnetic stirring at RT, 4 ml of hu2H11R35R74 (14.36 mg/ml in buffer A) are added, then 7.5 ml of buffer A, 1.45 ml of HEPES 1M, 1.05 ml of DMA, followed by 0.39 ml of a 10 mM DMA solution of L-DM4-AcNMe-PEG4-CONHS activated ester. After 30 min at RT, an extra 0.19 ml of 10 mM DMA solution of L-DM4-AcNMe-PEG4-CONHS activated ester is added. After 3 hrs at RT, the crude conjugation medium is diluted with 65 ml of HGS buffer and purified by TFF on Pellicon 3 cassette. The sample is diafiltered against ˜10 sample volumes of HGS buffer and then collected. The TFF tank and lines are washed with an extra 10 ml of HGS buffer. The two solutions are mixed, concentrated on Amicon 15 and filter-sterilized through 0.22 μm PVDF. 8.5 ml of hu2H11R35R74-PEG4-NMeAc-DM4 conjugate (c=6.01 mg/ml) was thus obtained. The conjugate is then analyzed for final drug load and monomeric purity. SEC analysis (H): DAR (SEC)=5.5; RT=16.7 min; monomeric purity=99.4%; HRMS data: see FIG. 2.

### 2.2. Preparation of L-DM4-AcNMe-PEG4-CONHS Activated Ester

Under magnetic stirring at RT, 133.4 mg of L-DM4 are introduced in a glass vial. A solution of 85 mg of 3-{2-[2-(2-{2-[(2-bromo-acetyl)-methyl-amino]-ethoxy}-ethoxy)-ethoxy]-ethoxy}-propionic acid 2,5-dioxo-pyrrolidin-1-yl ester in 0.2 ml of DMA is then added, followed by 32.9 μl of DIEA. After 1 hours at RT, the reaction medium is purified by flash-chromatography on 30 g of C18-grafted silica gel (gradient of elution water:acetonitrile 95:5 to 5:95 by volume). After concentration of fractions containing the desired product under RP, 71.3 mg of L-DM4-AcNMe-PEG4-CONHS activated ester are obtained in the form of a colourless glass-like product. Mass spectra (D); RT=0.98 min; [M+H−H₂O]+: m/z 1178 (main signal); [M+Na]+: m/z 1218; [M−H+HCO₂H]−: m/z 1240 ; ¹H NMR (500 MHz, δ in ppm, chloroform-d): 0.81 (s, 3H); 1.20 to 1.33 (m, 13H); 1.42 to 1.52 (m, 1H); 1.56 to 1.61 (m, 1H); 1.65 (s, 3H); 1.73 to 1.83 (m, 1H); 1.96 to 2.04 (m, 1H); 2.19 (dd, J=2.8 and 14.4 Hz, 1H); 2.29 to 2.41 (m, 1H); 2.55 to 2.66 (m, 2H); 2.83 to 2.93 (m, 12H); 3.04 (d, J=9.8 Hz, 1H); 3.12 (d, J=12.7 Hz, 1H); 3.18 to 3.25 (m, 5H); 3.37 (s, 3H); 3.47 to 3.54 (m, 3H); 3.57 to 3.68 (m, 15H); 3.85 (t, J=6.6 Hz, 2H); 3.99 (s, 3H); 4.29 (m, 1H); 4.79 (dd. J=2.8 and 12.2 Hz, 1H); 5.41 (q, J=6.7 Hz, 1H); 5.68 (dd. J=9.3 and 15.2 Hz, 1H); 6.23 (s, 1H); 6.43 (dd, J=11.0 and 15.2 Hz, 1H); 6.66 (s, 1H); 6.74 (d, J=11.0 Hz, 1H); 6.83 (s, 1H).

### 2.3. Preparation of 3-{2-[2-(2-{2-[(2-bromo-acetyl)-methyl-amino]-ethoxy}-ethoxy)-ethoxy]-ethoxy}-propionic acid 2,5-dioxo-pyrrolidin-1-yl ester

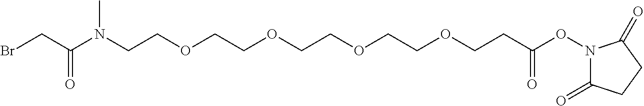

Under magnetic stirring, at RT, in a round bottom flask, 115.1 mg of 3-(2-{2-[2-(2-methylamino-ethoxy)-ethoxy]-ethoxy}-ethoxy)-propionic acid, 1.5 ml of DCM, 97.3 mg of bromo-acetic acid 2,5-dioxo-pyrrolidin-1-yl ester are successively introduced. After 2 h, 72 μl of DIEA are added, and after a further 1 hour at RT, 70.2 μl of DIC are added. The crude reaction medium is kept 4 hrs at RT, 16 hrs at −20° C., and then purified by flash-chromatography on 30 g of silica gel (gradient of elution DCM:methanol from 0:100 to 3:97 by volume). After concentration of fractions containing the desired product under RP, 85.8 mg of 3-{2-[2-(2-{2-[(2-bromo-acetyl)-methyl-amino]-ethoxy}-ethoxy)-ethoxy]-ethoxy}-propionic acid 2,5-dioxo-pyrrolidin-1-yl ester are obtained in the form of a white solid. Mass spectra (A); RT=0.84 min; [M+H]+: m/z 1497/499

### 2.4. Preparation of 3-(2-{2-[2-(2-methylamino-ethoxy)-ethoxy]-ethoxy}-ethoxy)-propionic acid

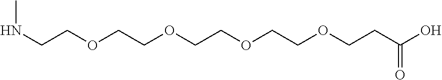

Under an inert atmosphere of argon, in a round bottom flask, with magnetic stirring, 120.1 mg of 3-[2-(2-{2-[2-(2,2,2-trifluoro-acetylamino)-ethoxy]-ethoxy}-ethoxy)ethoxy]-propionic acid methyl ester, 1 ml of anhydrous THF and 59.8 μl of CH₃I and successively introduced. The reaction medium is cooled with a ice/water bath at about 0° C. and 16.1 mg of NaH (50% pure in oil) is slowly added by small portions. After 15 min at 0° C., and 1 hr at RT, the crude reaction medium is concentrated to dryness under RP, and diluted with 0.5 ml of THF and 0.8 ml of water. At RT, 30.6 mg of LiOH is then added to the reaction medium. The crude reaction medium is kept 2 hrs at RT, 16 hrs at −20° C., and then purified by flash-chromatography on 30 g of C18-grafted silica gel (gradient of elution water:acetonitrile from 95:5 to 5:95 by volume). After concentration of fractions containing the desired product under RP, 115.3 mg of 3-(2-{2-[2-(2-methylamino-ethoxy)ethoxy]-ethoxy}-ethoxy)-propionic acid are obtained in the form of a yellow oil.

### 2.5. Preparation of 3-[2-(2-{2-[2-(2,2,2-trifluoro-acetylamino)-ethoxy]-ethoxy}-ethoxy)-ethoxy]-propionic acid methyl ester

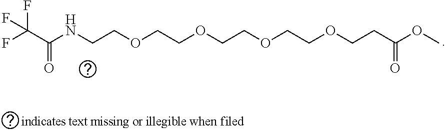

Under an inert atmosphere of argon, in a round bottom flask, with magnetic stirring, 230 mg of 3-(2-{2-[2-(2-amino-ethoxy)-ethoxy]-ethoxy}-ethoxy)-propionic acid (CA(PEG)₄, Pierce) 2 ml of DCM and 1 ml of methanol are successively introduced. At RT, 1 ml of trimethylsilyldiazomethane (2M solution in hexane) is slowly added to the reaction medium. After 2 hrs at RT, the excess of trimethylsilyldiazomethane is neutralized by addition of acetic acid. The crude is then evaporated to dryness under RP. The residue obtained is diluted with 2 ml of DCM, cooled to 0° C. with a water-ice bath, then 363 μl of TEA and 300 μl of TFAA are successively added. After 2 hrs 30 min at RT and 19 hrs at −20° C., 363 μl of TEA and 300 μl of TFAA are successively added. After 4 hrs 30 min at RT and the crude is stocked at −20° C. and then purified by flash-chromatography on 30 g of C18-grafted silica gel (gradient of elution water:acetonitrile from 95:5 to 5:95 by volume). After concentration of fractions containing the desired product under RP, 123 mg of 3-[2-(2-{2-[2-(2,2,2-trifluoro-acetylamino)-ethoxy]-ethoxy}-ethoxy)-ethoxy]-propionic acid methyl ester are obtained in the form of a pale-yellow oil. Mass spectra (A); RT=0.90 min; [M+H]+: m/z 376; [M−H]−: m/z 374.

### Example 3

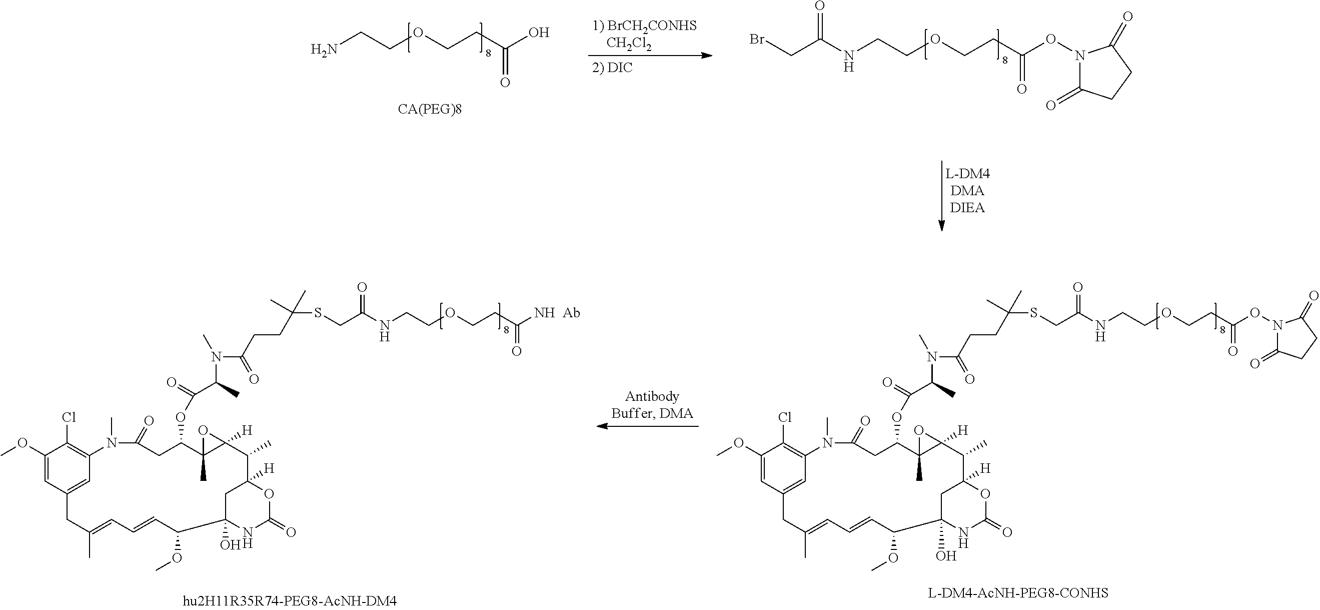

### 3.1. Preparation of Conjugate hu2H11R35R74-PEG8-NHAc-DM4

Under magnetic stirring at RT, 4 ml of hu2H11R35R74 (14.36 mg/ml in buffer A) are added, then 7.5 ml of buffer A, 1.45 ml of HEPES 1M, 1.05 ml of DMA, followed by 0.405 ml of a mM DMA solution of L-DM4-AcNMe-PEG-CONHS activated ester. After 30 min at RT, an extra 0.1 ml of 10 mM DMA solution of L-DM4-AcNMe-PEG8-CONHS activated ester is added. After 1 hr 45 min at RT, the crude conjugation medium is diluted with 60 ml of HGS buffer and purified by TFF on Pellicon 3 cassette. The sample is diafiltered against ˜1 sample volumes of HGS buffer and then collected. The TFF tank and lines are washed with an extra 10 ml of HGS buffer. The two solutions are mixed, concentrated on Amicon 15 and filter-sterilized through 0.22□m PVDF. 7.0 ml of hu2H11R35R74-PEG8-AcNMe-DM4 conjugate (c=6.95 mg/ml) was thus obtained. The conjugate is then analyzed for final drug load and monomeric purity. SEC analysis (H): DAR (SEC)=5.0; RT=16.593 min; monomeric purity=99.5%: HRMS data: see FIG. 3.

### 3.2. Preparation of L-DM4-AcNH-PEG8-CONHS Activated Ester

Under magnetic stirring at RT, 65 mg of 3-{2-[2-(2-{2-[2-(2-{2-[2-(3-bromo-propionylamino)-ethoxy]-ethoxy}-ethoxy)-ethoxy]-ethoxy}-ethoxy)-ethoxy]-ethoxy}-propionic acid 2,5-dioxo-pyrrolidin-1-yl ester are introduced in a glass vial, followed by a solution of 67.7 mg of L-DM4 in 0.85 ml of DMA and 16.5 μl of DIEA. After 48 hrs at RT, the reaction medium is purified by flash-chromatography on 10 g of silica gel (gradient of elution DCM:MeOH 100:0 to 90:10 by volume). After concentration of fractions 18 to 26 under RP, 17 mg of L-DM4-AcNH-PEG8-CONHS activated ester are obtained in the form of a colourless glass. Mass spectra (B): RT=4.08 min; [M+H−H₂O]+: m/z 1340 (main signal); [M+Na]+: m/z 1380: [M−H+HCO₂H]−: m/z 1402: ¹H NMR (400 MHz, δ in ppm, chloroform-d): 0.81 (s, 3H); 1.22 (s, 3H); 1.23 (s, 3H); 1.26 (m, 1H); 1.30 (d, J=6.8 Hz, 6H); 1.41 to 1.52 (m, 1H); 1.65 (s, 3H); 1.80 (m, 1H); 1.89 to 1.99 (m, 1H); 2.19 (m, 1H); 2.37 (m, 1H); 2.47 to 2.67 (m, 2H); 2.81 to 2.93 (m, 10H); 2.99 (d, J=16.6 Hz, 1H); 3.04 (d, J=9.8 Hz, 1H); 3.16 (d broad, J=13.7 Hz, 1H); 3.23 (s, 3H); 3.32 (s broad, 1H); 3.37 (s, 3H); 3.44 (m, 2H); 3.51 (d, J=9 Hz, 1H); 3.54 (t, J=5.4 Hz, 2H); 3.59 to 3.73 (m, 29H); 3.86 (t, J=6.6 Hz, 2H); 4.00 (s, 3H); 4.22 to 4.33 (m, 1H); 4.78 (dd, J=2.9 and 12.2 Hz, 1H); 5.43 (q, J=6.8 Hz, 1H); 5.67 (dd, J=9.0 and 15.2 Hz, 1H); 6.23 (s, 1H); 6.44 (dd, J=11.2 and 15.2 Hz, 1H); 6.65 (d, J=1.5 Hz, 1H); 6.75 (d, J=11.2 Hz, 1H); 6.86 (d, J=1.5 Hz, 1H); 7.02 to 7.13 (m, 1H).

### 3.3. Preparation of 3-{2-[2-(2-{2-[2-(2-{2-[2-(3-bromo-propionylamino)-ethoxy]-ethoxy}-ethoxy)-ethoxy]-ethoxy}-ethoxy)-ethoxy]-ethoxy}-propionic acid 2,5-dioxo-pyrrolidin-1-yl ester

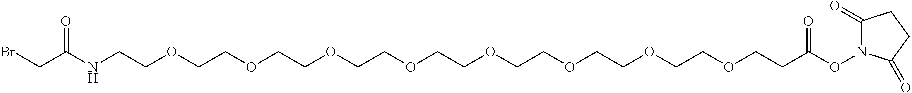

Under magnetic stirring at RT, 100 mg of 3-(2-{2-[2-(2-amino-ethoxy)-ethoxy]-ethoxy}-ethoxy)-propionic acid (CA(PEG)₄, Pierce), 2 ml of DCM and 53.5 mg of bromo-acetic acid 2,5-dioxo-pyrrolidin-1-yl ester are successively introduced in a glass vial. After 1 hr at RT, 35.1 μl of DIC are added. After 1 hr, the crude reaction medium is filtered on sintered glass, concentrated to dryness under RP, dilute with 10 ml of AcOEt, filtered on sintered glass and concentrated to dryness under RP. 76.5 mg of 3-{2-[2-(2-{2-[2-(2-{2-[2-(3-bromo-propionylamino)-ethoxy]-ethoxy}-ethoxy)-ethoxy]-ethoxy}-ethoxy)-ethoxy]-ethoxy}-propionic acid 2,5-dioxo-pyrrolidin-1-yl ester are obtained in the form of a colourless oil. Mass spectra (A); RT=0.80 min; [M+H]+: m/z 659/661; [M−H+HCO₂H]−: m/z 703/705

### Example 4

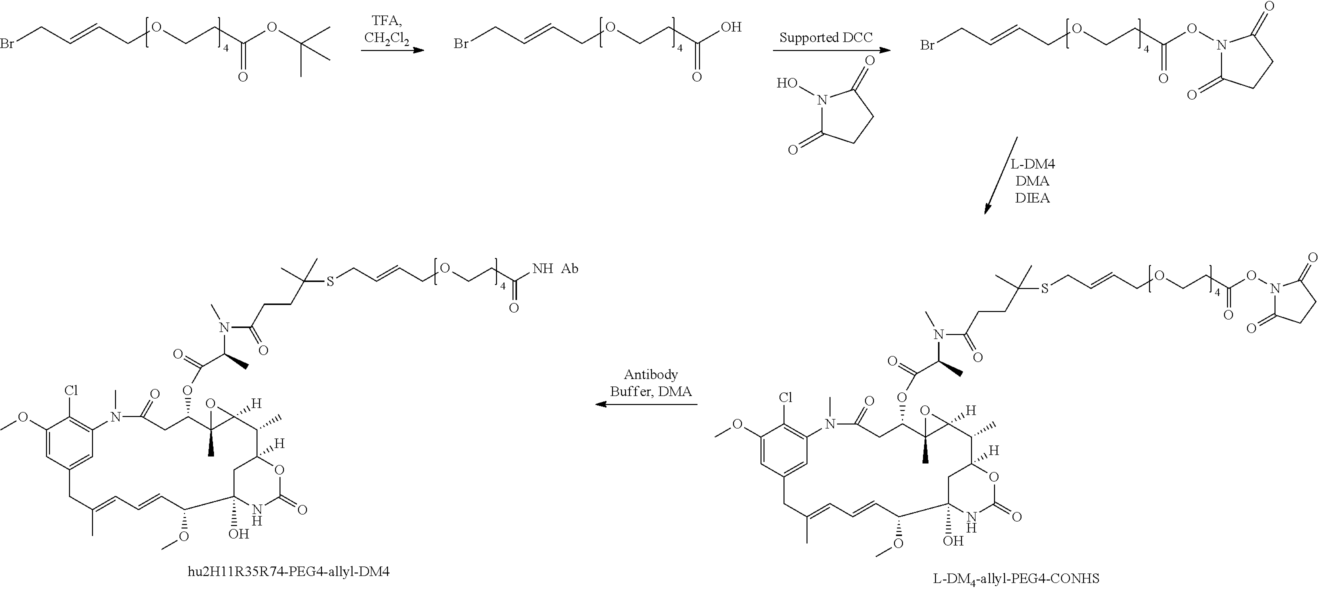

### 4.1. Preparation of Conjugate hu2H11R35R74-PEG4-Allyl-DM4

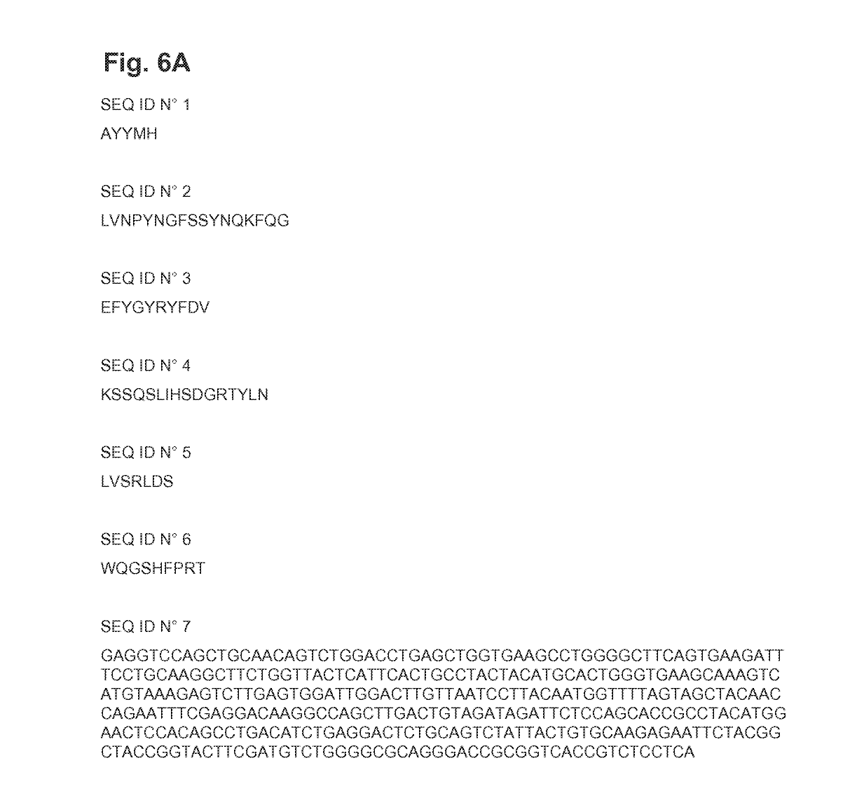

Under magnetic stirring at RT, 4 ml of hu2H11R35R74 (14.36 mg/ml in buffer A) are added, then 7.5 ml of buffer A, 1.45 ml of HEPES 1M, 1.14 ml of DMA, followed by 0.3 ml of a 10 mM DMA solution of L-DM4-Allyl-PEG4-CONHS activated ester. After 30 min at RT, an extra 0.125 ml of 10 mM DMA solution of L-DM4-Allyl-PEG4-CONHS activated ester is added. After 1 hr 25 min at RT, the crude conjugation medium is diluted with 65 ml of HGS buffer and purified by TFF on Pellicon 3 cassette. The sample is diafiltered against ˜10 sample volumes of HGS buffer and then collected. The TFF tank and lines are washed with an extra 10 ml of HGS buffer. The two solutions are mixed, concentrated on Amicon 15 and filter-sterilized through 0.22 μm PVDF. 8.0 ml of hu2H11R35R74-PEG4-Allyl-DM4 conjugate (c=5.22 mg/ml) was obtained. The conjugate is then analyzed for final drug load and monomeric purity. SEC analysis (H): DAR (SEC)=5.3; RT=16.767 min; monomeric purity=99.4%; HRMS data: see FIG. 4.

### 4.2. Preparation of L-DM4-Allyl-PEG4-CONHS Activated Ester

Under magnetic stirring at RT, 70 mg of L-DM4, 45 mg of 3-(2-{2-[2-(4-bromo-but-2-enyloxy)-ethoxy]-ethoxy}-ethoxy)-propionic acid 2,5-dioxo-pyrrolidin-1-yl ester (Bromo-Allyl-PEG₄-CONHS), 0.5 ml of DMA and 23.5 μl of DIEA are successively introduced in a glass vial. After 2 hrs at RT and 17 hrs at −20° C. 50 μl of DIEA is added. After 24 hrs at RT, the reaction medium is purified by flash-chromatography on 30 g of C-18 grafted silica gel (gradient of elution water:acetonitrile 95:5 to 5:95 by volume). After concentration of fractions containing the expected product under RP, 47.1 mg of L-DM4-Allyl-PEG4-CONHS activated ester are obtained in the form of a white solid. Mass spectra (D); RT=1.06 min; [M+Na]+: m/z 1173: ¹H NMR (500 MHz, δ in ppm, chloroform-d): 0.81 (s, 3H); 1.18 to 1.39 (m, 13H); 1.42 to 1.52 (m, 1H); 1.58 (d, J=13.4 Hz, 1H); 1.65 (s, 3H), 1.73 to 1.82 (m, 1H); 1.86 to 1.95 (m, 1H); 2.19 (d, J=14.3 Hz, 1H); 2.40 (m, 1H); 2.51 to 2.65 (m, 2 H); 2.82 to 2.95 (m, 9H); 2.98 to 3.07 (m, 2H); 3.12 (d, J=12.6 Hz, 1H); 3.18 to 3.27 (m, 1H); 3.23 (s, 3H); 3.36 (s, 3H); 3.51 (d, J=9.1 Hz, 1H); 3.54 to 3.82 (m, 13H); 3.86 (t, J=6.4 Hz, 2H); 3.91 to 3.95 (m, 2H); 3.99 (s, 3H); 4.28 (t, J=11.0 Hz, 1H); 4.78 (dd, J=2.6 and 11.9 Hz, 1H); 5.44 (q, J=6.7 Hz, 1H); 5.49 to 5.63 (m, 2H); 5.68 (dd, J=9.1 and 15.0 Hz, 1H); 6.24 (s, 1H); 6.43 (dd, J=11.1 and 15.0 Hz, 1H); 6.66 (s, 1H); 6.77 (d, J=11.1 Hz, 1H); 6.83 (s, 1H).

### 4.3. Preparation of 3-(2-{2-[2-(4-bromo-but-2-enyloxy)-ethoxy]-ethoxy}-ethoxy)-propionic acid 2,5-dioxo-pyrrolidin-1-yl ester

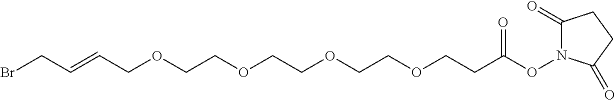

At RT, 200 mg of 3-(2-{2-[2-(4-bromo-but-2-enyloxy)-ethoxy]-ethoxy}-ethoxy)-propionic acid, 4 ml of DCM and 232.3 mg of supported DCC (2 equivalents) are successively introduced in a glass vial. After 1 hr at RT, 64.8 mg of NHS are added. After 5 hrs at RT, the crude is filtered on sintered glass, solids are washed with DCM, and the combined filtrates are concentrated to dryness under RP. Purification by flash-chromatography on 15 g of silica gel (gradient of elution MeOH:DCM 0:100 to 10:90 by volume), and concentration of fractions containing the expected product under RP, afforded 46 mg of 3-(2-{2-[2-(4-bromo-but-2-enyloxy)-ethoxy]-ethoxy}-ethoxy)-propionic acid 2,5-dioxo-pyrrolidin-1-yl ester (Bromo-Allyl-PEG4-CONHS) are obtained in the form of a pale yellow oil. Mass spectra (A); RT=1.02 min; [M+H]+: m/z 454/456: [M+Na]+: m/z 476/478; [M−H+HCO₂H]−: m/z 498/500.

### 4.4. Preparation of 3-(2-{2-[2-(4-bromo-but-2-enyloxy)-ethoxy]-ethoxy}-ethoxy)-propionic acid

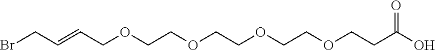

At RT, a solution of 1 g of 3-(2-{2-[2-(4-bromo-but-2-enyloxy)-ethoxy]-ethoxy}-ethoxy)-propionic acid tert-butyl ester (commercially available), 6 ml of TFA and 3 ml of DCM is stirred during 3 hrs, and then concentrated to dryness under RP. The oily residue is diluted with toluene and concentrated to dryness under RP affording 853 mg of 3-(2-{2-[2-(4-bromo-but-2-enyloxy)-ethoxy]-ethoxy}-ethoxy)-propionic acid in the form of a brown oil.

### Example 5

### 5.1. Preparation of conjugate hu2H11-PEG4-NHAc-DM4

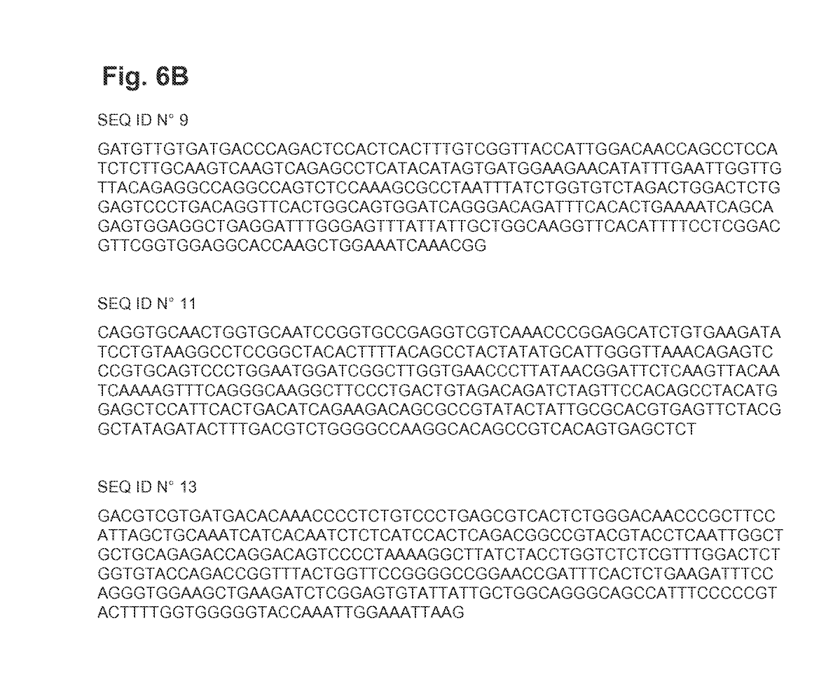

Conjugate hu2H11-PEG4-NHAc-DM4 could be prepared in a manner similar to example 1: under stirring, at RT, 1 ml of hu2H11 (8.52 mg/ml in buffer A) is added, then 0.7 ml of buffer A, 0.213 ml of HEPES 1M, 0.7 ml of DMA, followed by 0.085 ml of a 10 mM DMA solution of L-DM4-AcNH-PEG4-CONHS activated ester diluted with 0.128 ml of DMA. After 2 hrs at RT, the crude medium is concentrated on Amicon 4 at 7000 G, buffer exchanged with HGS buffer on Nap-10 column, and finally purified on a 5 ml Zeba column. 1.15 ml of hu2H11-PEG4-NHAc-DM4 conjugate (c=3.78 mg/ml) was thus obtained. The conjugate is analyzed for final drug load and monomeric purity. SEC analysis (method H): DAR (UV)=6.6: DAR (SEC)=5.6; RT=15.387 min; monomeric purity=99.7%: HRMS data; see FIG. 5.

Likewise, other conjugates involving hu2H11 and being described in examples 6-8 were prepared (see Table IIa).

### Example 9

### Inhibition of Growth of MDA-MB231 Tumor Cells (from ECAAC Ref. #92020424)

Cells in exponential phase of growth were trypsinized and resuspended in appropriate culture medium (DMEM/F12 Gibco #21331; 10% SVF Gibco #10500-056; 2 nM Glutamine Gibco #25030). Cell suspension was distributed in 96-well Cytostar culture plates (GE Healthcare Europe, #RPNQ0163) in complete serum-containing media at a density of 5000 cells/well. After coating for 4 hrs, serial dilutions of conjugates were added to triplicate wells at concentrations ranging between 10⁻⁷ and 10⁻¹² M. Cells were cultured at 37° C./5% CO₂ in the presence of the conjugates for 3 days. The 4^{th} day, 10 μl of a solution of ¹⁴C-thymidine (0.1 μCi/well (Perkin Elmer #NEC56825000)) was added to each well. The uptake of ¹⁴C-thymidine was measured 96 hrs after the experiment has been started with a microbeta radioactive counter (Perkin Elmer). Cell-free reagent blanks were subtracted from the test well readings and the data were plotted as surviving fractions obtained by dividing readings of the conjugate-treated cells by the average of readings from control wells of vehicle-treated cells. In these experiments, the naked antibody (hu2H11 or hu2H11R35R74) was added to the wells at a concentration of 1 μM at the beginning of the experiment, and inhibition of proliferation was measured as previously described.

Results reported in Tables IIa and IIb suggest that the conjugates display strong in vitro proliferation inhibition properties on MDA-MB231 cells and act through binding to the antigen according to the competition study run in the presence of naked hu2H11 or/hu2H11R35R74.

TABLE IIa

| | | IC₅₀ [pM] | | |
|---|---|---|---|---|
| | DAR | conjugate | +naked | |
| conjugate | (UV) | alone | hu2H11 | ratio |
| hu2H11-PEG4-NHAc-DM4 (see | 6.6 | 895 | 75223 | 84 |
| ex. 1.1) | | | | |
| hu2H11-PEG8-NHAc-DM4 | 5.6 | 1007 | 20726 | 21 |
| hu2H11-PEG4-Allyl-DM4 | 5.1 | 437 | 25907 | 59 |
| hu2H11-PEG4-NMeAc-DM4 | 4.1 | 1400 | 85379 | 61 |

TABLE IIb

| | | IC₅₀ [pM] | | |
|---|---|---|---|---|
| | DAR | conjugate | +naked | |
| conjugate | (UV) | alone | hu2H11R35R74 | ratio |
| hu2H11R35R74-PEG4- | 5.9 | 147 | 29731 | 202 |
| NHAc-DM4 | | | | |
| hu2H11R35R74-PEG8- | 4.9 | 400 | 24955 | 62 |
| NHAc-DM4 | | | | |
| hu2H11R35R74-PEG4- | 5.3 | 161 | 7820 | 49 |
| Allyl-DM4 | | | | |
| hu2H11R35R74-PEG4- | 5.4 | 217 | 36400 | 168 |
| NMeAc-DM4 | | | | |

### Example 10

### L-DM4-AcNH-PEG₄-COOMe

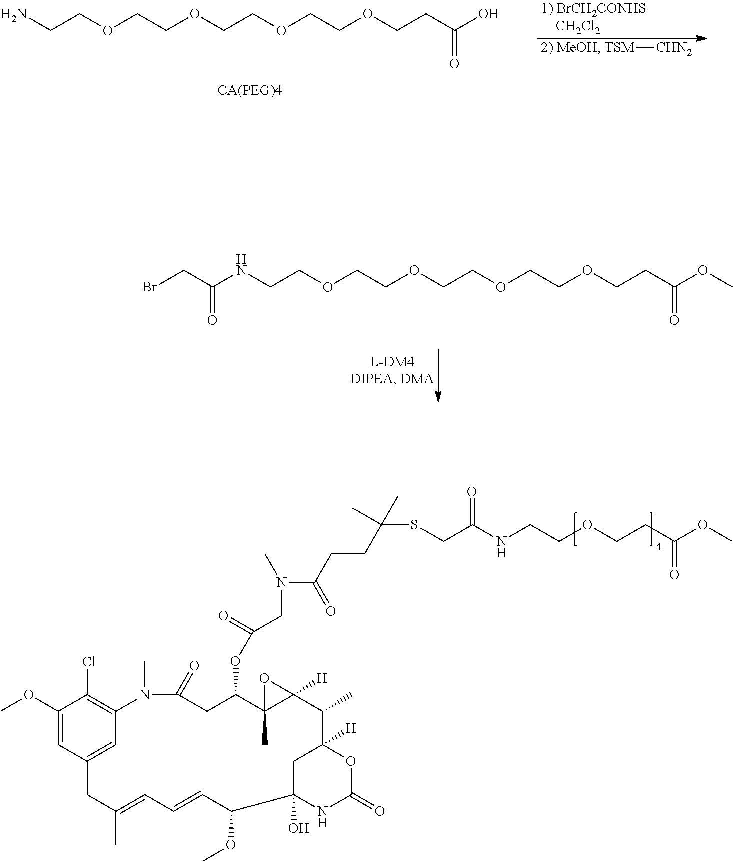

### 10.1. Preparation of Free-Drug L-DM4-AcNH-PEG₄-COOMe

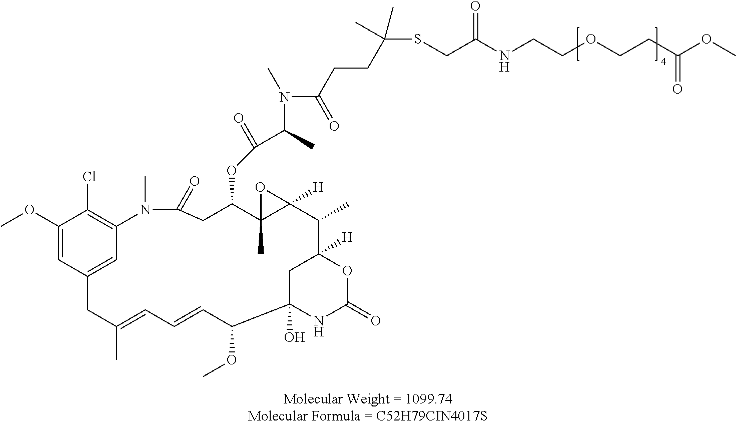

Under magnetic stirring, at RT, under an inert atmosphere of Ar, in a glass vial, 57.4 mg of 3-[2-(2-{2-[2-(2-bromo-acetylamino)-ethoxy]-ethoxy}-ethoxy)-ethoxy]-propionic acid methyl ester, a solution of 80 mg of L-DM4 in 0.44 ml of DMA, finally and 19.6 μl of DIPEA are successfully introduced. After 18 hrs at RT, the crude is diluted with 10 ml of water, extracted with 3×7 ml of AcOEt. Organic phases are gathered, dried over MgSO₄, filtered and concentrated to dryness under RP. 124 mg of a colourless oil are obtained, which product is diluted with a minimum amount of DMA and purified by flash chromatography on C18-grafted silica gel (Merck, C18, 5 g, 25-40 μm, 18 ml/min, gradient of elution water:acetonitrile 100:0 to 5:95 by volume). After concentration of fractions containing the expected compound under RP, 12.8 mg of the methyl ester L-DM4-AcNH-PEG₄-COOMe are obtained in the form of a colourless film. Mass spectra (C): t_{R}=0.97 min; [M+H]+: m/z 1099: [M−H]−: m/z 1097; ¹H NMR (500 MHz, in ppm, chloroform-d): 0.80 (s, 3H); 1.21 (s, 3H); 1.22 (s, 3H); 1.24 to 1.35 (m, 7H); 1.46 (td, J=6.4 and 10.2 Hz, 1H); 1.57 (d, J=13.4 Hz, 1H); 1.64 (s, 3 H); 1.80 (ddd, J=4.9 and 11.5 and 14.6 Hz, 1H); 1.92 (m, 1H); 2.18 (dd, J=2.3 et 14.7 Hz, 1H); 2.36 (ddd, J=4.8 and 11.4 and 16.2 Hz, 1H); 2.52 (ddd, J=5.1 and 11.3 and 16.3 Hz, 1H); 2.59 (t, J=6.4 Hz, 2H); 2.63 (m, 1H); 2.86 (s, 3H); 2.88 (m, 1H); 2.98 (d, J=16.7 Hz, 1H); 3.03 (d, J=9.6 Hz, 1H); 3.15 (d, J=12.6 Hz, 1H); 3.23 (s, 3H); 3.36 (s, 3H); 3.42 (m, 2H); 3.50 (d, J=9 Hz, 1H); 3.54 (m, 2H); 3.63 (m, 13H); 3.68 (s, 3H); 3.75 (t, J=6.4 Hz, 2H); 3.99 (s, 3H); 4.27 (1, J=11.3 Hz, 1H); 4.78 (dd, J=2.2 et 12.1 Hz, 1H); 5.42 (q, J=6.9 Hz, 1H); 5.66 (dd, J=9.1 and 15.4 Hz, 1H); 6.21 (s, 1H); 6.43 (dd, J=11.0 and 15.4 Hz, 1H); 6.64 (s, 1H); 6.74 (d, J=11.0 Hz, 1H); 6.85 (s, 1H); 7.05 (t, J=5.5 Hz, 1H).

### 10.2. Preparation of 3-[2-(2-{2-[2-(2-bromo-acetylamino)-ethoxy]-ethoxy}-ethoxy)-ethoxy]-propionic acid methyl ester

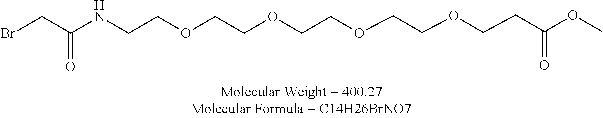

Under magnetic stirring, at RT, 100 mg of 3-(2-{2-[2-(2-amino-ethoxy)-ethoxy]-ethoxy}-ethoxy)-propionic acid (CA(PEG)₄, Pierce), 89 mg of bromo-acetic acid 2,5-dioxo-pyrrolidin-1-yl ester and 2 ml of DCM are successively introduced in a glass vial. After 1 hr at RT, 0.7 ml of MeOH and 0.38 ml of a 2 M trimethylsilydiazomethane solution in hexane are added. After 1 hr at RT, the crude reaction mixture is concentrated to dryness under RP, then diluted with a minimum amount of DMA and purified by flash chromatography on C18-grafted silica gel (Merck, C18, 5 g, 25-40 μm, 18 ml/min, gradient of elution water:acetonitrile 100:0 to 5:95 by volume). After concentration of fractions containing the expected compound under RP, 58 mg of 3-[2-(2-{2-[2-(2-bromo-acetylamino)-ethoxy]-ethoxy}-ethoxy)-ethoxy]-propionic acid methyl ester are obtained in the form of a colourless oil. Mass spectra (A): tᵣ=0.75 min; [M+H]+: m/z 400/402

Bromo-acetic acid 2,5-dioxo-pyrrolidin-1-yl ester could be prepared following published protocol (Biochemistry 1974, 481).

### Example 11

### L-DM4-AcNMe-PEG₄-COOMe

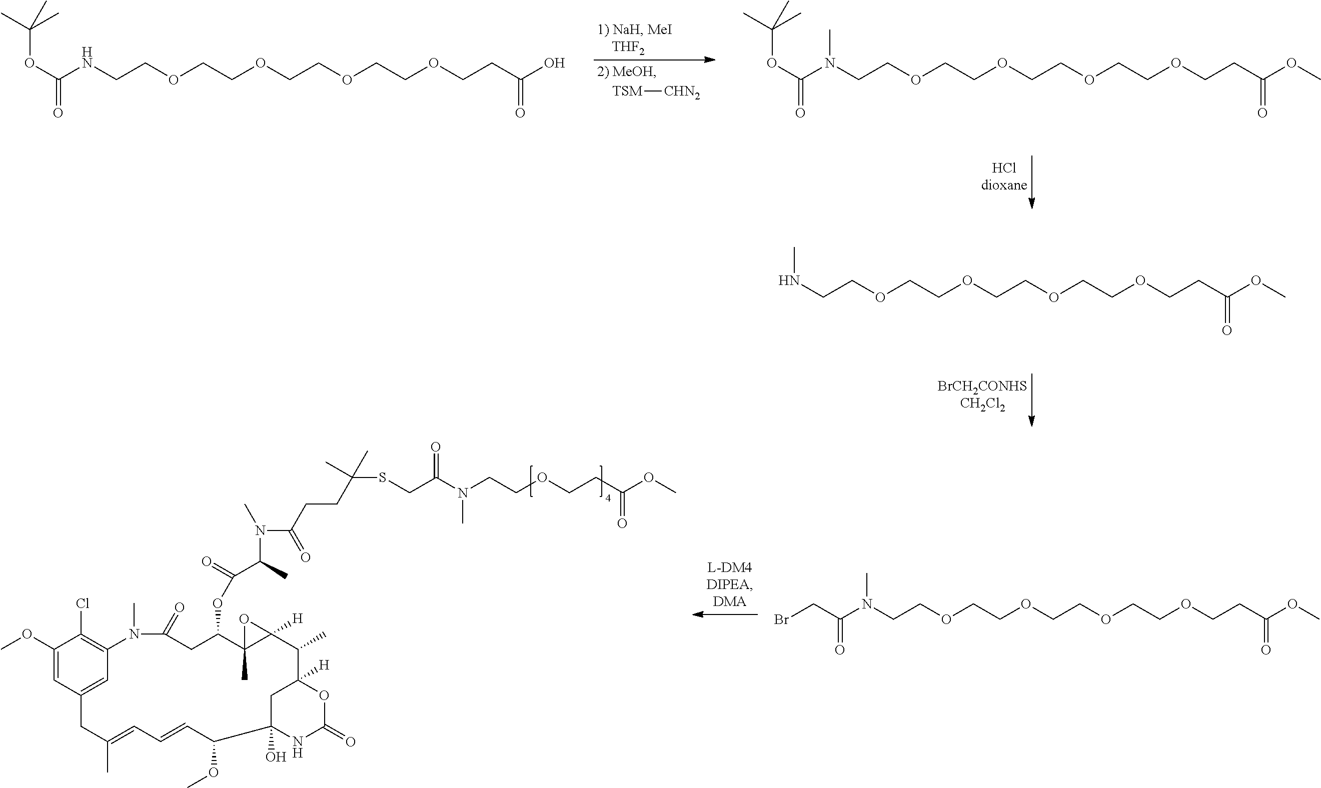

### 11.1. Preparation of Free-Drug L-DM4-AcNMe-PEG₄-COOMe

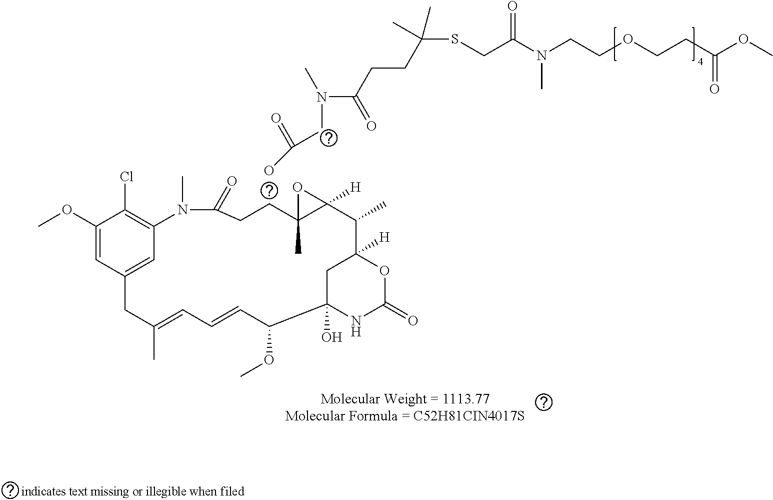

Under magnetic stirring, at RT, in a glass vial, 30 mg of L-DM4, a solution of 20.8 mg of 3-{2-[2-(2-{2-[(2-bromo-acetyl)-methyl-amino]-ethoxy}-ethoxy)-ethoxy]-ethoxy}-propionic acid methyl ester in 0.3 ml of DMA, and 7.4 μl of DIPEA are successfully introduced. After 18 hrs at RT, the reaction medium is diluted with 7 ml of AcOEt and washed twice with 5 ml of water. The organic phase is washed with brine, dried over MgSO₄, filtered and concentrated to dryness under RP. 39 mg of a colourless glass are obtained, which product is diluted with a minimum amount of DMA/MeOH mixture and purified by chromatography on CIS-grafted silica gel (XTerra® C18, 5 μm, 50×30 mm, 30 ml/min, gradient of elution water:acetonitrile 95:5 to 5:95 by volume). After concentration of fractions containing the expected compound under reduce pressure, 7.8 nm of the methyl ester L-DM4-AcNMe-PEG₄-COOMe are obtained in the form of a colourless solid. Mass spectra (C): t_{R}=1.00 min; [M+H−H₂O]+: m/z 1095: [M+Na⁺]+: m/z 1135; [M−H+HCO₂H]−: m/z 1157; [M−H]−: m/z 1111. ¹H NMR (500 MHz, δ in ppm, DMSO-d6): 0.78 (s, 3H); 1.12 (d, J=6.6 Hz, 3H); 1.16 (m, 9H); 1.26 (m, 1H); 1.40 to 1.51 (m, 2H); 1.59 (s, 3H); 1.62 (m, 1H); 1.87 (m, 1H); 2.04 (m, 1H); 2.28 (m, 1H); 2.47 to 2.58 (m partially masked, 4H); 2.73 (s, 3H); 2.76 (s, 1H); 2.80 (d, J=9.6 Hz, 1H); 2.95 (s, 2H); 3.10 (s, 3H); 3.16 to 3.48 (m partially masked, 21H); 3.25 (s, 3H); 3.59 (s, 3H); 3.63 (t, J=6.4 Hz, 2H); 3.93 (s, 3H); 4.08 (m, 1H); 4.53 (dd, J=2.9 to 11.9 Hz, 1H); 5.32 (q, J=6.6 Hz, 1H); 5.58 (dd, J=9.3 to 15.1 Hz, 1H); 5.89 (s, 1H); 6.49 to 6.58 (m, 2H) 6.62 (m, 1H); 6.84 (s, 1H); 7.19 (s, 1H).

### 11.2. Preparation of 3-{2-[2-(2-{2-[(2-bromo-acetyl)-methyl-amino]-ethoxy}-ethoxy)-ethoxy]-ethoxy}-propionic acid methyl ester

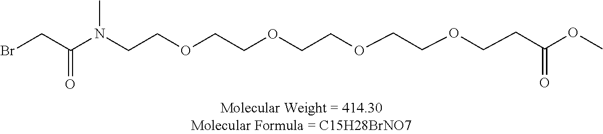

Under magnetic stirring, at RT, under an inert atmosphere of Ar, 127 mg of 3-(2-{2-[2-(2-methylamino-ethoxy)-ethoxy]-ethoxy}-ethoxy)-propionic acid methyl ester, 102.2 mg of bromo-acetic acid 2,5-dioxo-pyrrolidin-1-yl ester and 1.2 ml of DCM are successfully added. After 45 min, the crude is concentrated under RP and purified by flash-chromatography on 5 g of CN-grafted silica gel (gradient of elution n.heptane/iPrOH/AcOEt with increasing iPrOH portion). After concentration of fractions containing the expected compound under RP, 122 mg of 3-{2-[2-(2-{2-[(2-bromo-acetyl)-methyl-amino]-ethoxy}-ethoxy)-ethoxy]-ethoxy}-propionic acid methyl ester are obtained in the form of a colourless oil. Mass spectra (A): t_{R}=0.84 min; [M+H]+: m/z 414/416.

Bromo-acetic acid 2,5-dioxo-pyrrolidin-1-yl ester could be prepared following published protocol (Biochemistry 1974, 481).

### 11.3. Preparation of 3-(2-{2-[2-(2-methylamino-ethoxy)-ethoxy]-ethoxy}-ethoxy)-propionic acid methyl ester

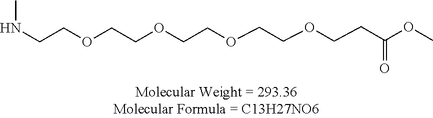

Under magnetic stirring, at RT, under an inert atmosphere of Ar, 271.7 mg of 3-[2-(2-{2-[2-(tert-butoxycarbonyl-methyl-amino)-ethoxy]-ethoxy}-ethoxy)-ethoxy]-propionic acid methyl ester are solubilised in 2 ml of hydrochloric acid 4N solution in dioxane. After 18 hrs, the crude reaction medium is concentrated under RP, solubilised in 4 ml of MeOH and passed through a 3 g SCX SPE column (conditioning with 10 ml MeOH, washing with 10 ml MeOH and elution with ammonia 2N in MeOH). After concentration of elution fraction under RP, 146 mg of colourless oil are obtained. This oil was dissolved in AcOEt, dried on MgSO₄, filtered and evaporated under RP. 127 mg of 3-(2-{2-[2-(2-methylamino-ethoxy)-ethoxy]-ethoxy}-ethoxy)-propionic acid methyl ester are obtained as a colourless oil. Mass spectra (A): t_{R}=0.40 min; [M+H]+: m/z 294.

### 11.4. Preparation of 3-[2-(2-{2-[2-(tert-butoxycarbonyl-methyl-amino)-ethoxy]-ethoxy}-ethoxy)-ethoxy]-propionic acid methyl ester

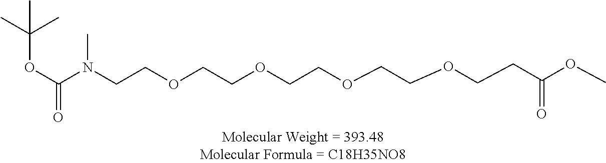

Under magnetic stirring, at RT, under an inert atmosphere of Ar, a solution of 227 mg of 3-[2-(2-{2-[2-(tert-butoxycarbonyl-methyl-amino)-ethoxy]-ethoxy}-ethoxy)-ethoxy]-propionic acid in 0.644 ml of DCM and 0.644 ml of MeOH is cooled down to about 0° C. with a water-ice-sodium chloride. 0.449 ml of a 2 M solution of trimethylsilydiazomethane in hexane are added, and after 17 hrs at RT, a 0.5 M solution of acetic acid in MeOH is added in order to achieve a pH between 5 and 6. The crude is diluted with AcOEt (30 ml), washed with water (2×15 ml), with brine (15 ml), and the organic phase is dried on MgSO₄, filtered and evaporated under RP. 209.7 mg of 3-[2-(2-{2-[2-(tert-butoxycarbonyl-methyl-amino)-ethoxy]-ethoxy}-ethoxy)-ethoxy]-propionic acid methyl ester are obtained as a colourless oil. Mass spectra (A): t_{R}=1.18 min; [M+H]+: m/z 294, 338, 394.

### 11.5. Preparation of 3-[2-(2-{2-[2-(tert-butoxycarbonyl-methyl-amino)-ethoxy]-ethoxy}-ethoxy)-ethoxy]-propionic acid

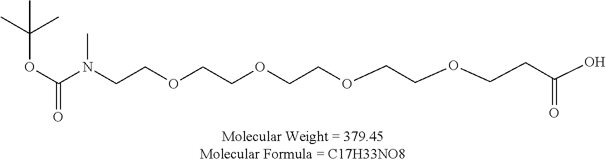

Under magnetic stirring, at RT, under an inert atmosphere of Ar, a solution of 330 mg of commercially available 3-(2-{2-[2-(2-tert-butoxycarbonylamino-ethoxy)-ethoxy]-ethoxy}-ethoxy)-propionic acid in 4 ml of THF is cooled down to about 0° C. with a water-ice-sodium chloride. 72.2 mg of NaH (70% in oil) are slowly added, and 25 min later 0.174 ml of MeI. Cooling bath is removed, and after 3 hrs at RT. 120 mg of NaH and 0.2 ml of MeI are added. After 1.5 hr at RT, a diluted aqueous solution of acetic acid is added in order to achieve a pH between 5 and 6. The crude reaction mixture is diluted with AcOEt (30 ml), washed with water (2×20 ml), with brine (10 ml), and the organic phase is dried on MgSO₄, filtered and evaporated under RP. 227 mg of 3-[2-(2-{2-[2-(tert-butoxycarbonyl-methyl-amino)-ethoxy]-ethoxy}-ethoxy)-ethoxy]-propionic acid are obtained as a colourless oil. Mass spectra (A): t_{R}=1.02 min; [M+H]+: m/z 280, 380.

### Example 12

### L-DM4-Allyl-PEG₄-COOMe

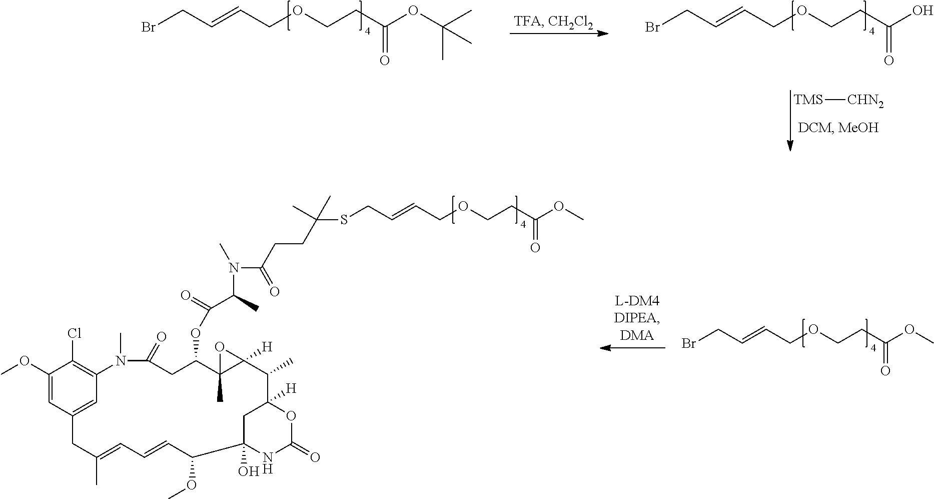

### 12.1. Preparation of Free-Drug L-DM14-Allyl-PEG₄-COOMe

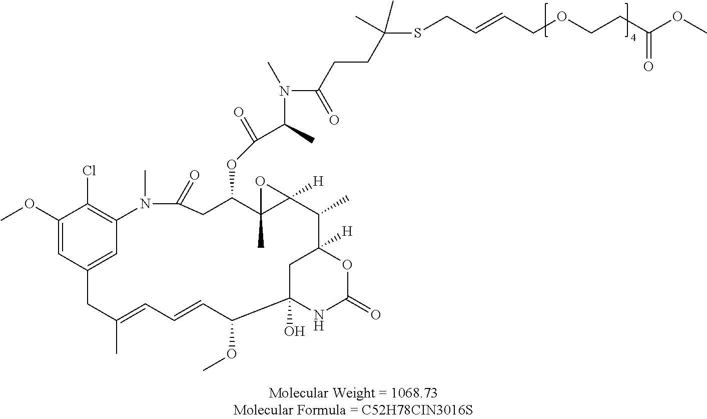

Under magnetic stirring, at RT, in a glass vial, 36.7 mg of L-DM4, a solution of 20.8 mg of 3-(2-{2-[2-(4-bromo-but-2-enyloxy)-ethoxy]-ethoxy}-ethoxy)-propionic acid methyl ester in 0.37 ml of DMA, and finally 9.4 μl of DIPEA are successfully introduced. After 1.5 hrs at RT, and 18 hrs at −18° C., the reaction medium is purified by flash chromatography on C18-grafted silica gel (Merck, C18, 5 g, 25-40 μm, 18 ml/min, gradient of elution water:acetonitrile 100:0 to 5:95 by volume). After concentration of fractions containing the expected compound under RP, 18.2 mg of a white solid are obtained and purified by flash-chromatography on CN-grafted silica gel (gradient of elution n.heptane/iPrOH/AcOEt with increasing AcOEt portion). 4.02 mg of the methyl ester L-DM4-Allyl-PEG₄-COOMe are obtained in the form of a white solid. Mass spectra (C): t_{R}=1.09 min; [M−H+HCOOH]−: m/z 1112; [M+Na]+: m/z 1090. ¹H NMR (500 MHz, in ppm, chloroform-d): 0.80 (s, 3H); 1.18 to 1.26 (m, 7H); 1.27 to 1.31 (m, 6H); 1.40 à 1.50 (m, 1H); 1.57 (d, J=13.7 Hz, 3H); 1.64 (s, 3H); 1.77 (ddd, J=4.8 and 11.7 and 14.5 Hz, 1H); 1.91 (ddd J=4.8 and 11.7 and 14.5 Hz, 1H); 2.18 (dd, J=2.5 and 14.3 Hz, 1H); 2.38 (m, 1H); 2.57 (m, 4H); 2.86 (s, 2H); 2.89 (m, 1H); 3.00 (m, 1H); 3.04 (d, J=9.9 Hz, 1H); 3.11 (d, J=12.3 Hz, 1H); 3.23 (s, 3H); 3.35 (s, 3H); 3.50 (d, J=9.1 Hz, 1H); 3.55 (m, 2H); 3.63 (m, 10H); 3.69 (s, 3H); 3.75 (t, J=6.4 Hz, 2H); 3.92 (d, J=5.2 Hz, 2H); 3.98 (s, 3H); 4.27 (ddd, J=1.6 and 10.4 and 12.3 Hz, 1H); 4.78 (dd, J=3.0 and 11.8 Hz, 1H); 5.43 (q, J=6.8 Hz, 1H); 5.48 à 5.61 (m, 2H); 5.67 (dd, J=9.2 and 15.2 Hz, 1H); 6.20 (d, J=0.5 Hz, 1H); 6.42 (dd, J=11.3 and 15.4 Hz, 1H); 6.65 (d, J=1.9 Hz; 1H); 6.77 (d, J=11.3 Hz, 1H); 6.82 (d, J=1.1 Hz, 1H).

### 12.2. Preparation of 3-(2-{2-[2-(4-bromo-but-2-enyloxy)-ethoxy]-ethoxy}-ethoxy)-propionic acid methyl ester

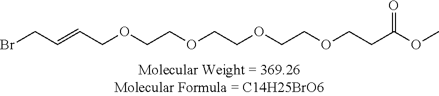

Under magnetic stirring, to a cooled solution (water-ice bath) of 50 mg of 3-(2-{2-[2-(4-bromo-but-2-enyloxy)-ethoxy]-ethoxy}-ethoxy)-propionic acid in 1 ml of DCM and 0.25 ml of MeOH, 0.11 ml of a 2M trimethylsilydiazomethane solution in hexane are added. After removing the water-ice bath, the crude reaction mixture is stirred 1.5 hr at RT, neutralized with 2 drops of acetic acid and concentrated to dryness under RP. After azeotropic evaporation with toluene, 47 mg of 3-(2-{2-[2-(4-bromo-but-2-enyloxy)-ethoxy]-ethoxy}-ethoxy)-propionic acid methyl ester are obtained in the form of an amber oil. Mass spectra (A): t_{R}=1.12 min; [M+H]+: m/z 369/371.

Preparation of 3-(2-{2-[2-(4-bromo-but-2-enyloxy)-ethoxy]-ethoxy}-ethoxy)-propionic acid has been described in example 4.

### Example 13

### L-DM4-AcNH-PEG-COOMe

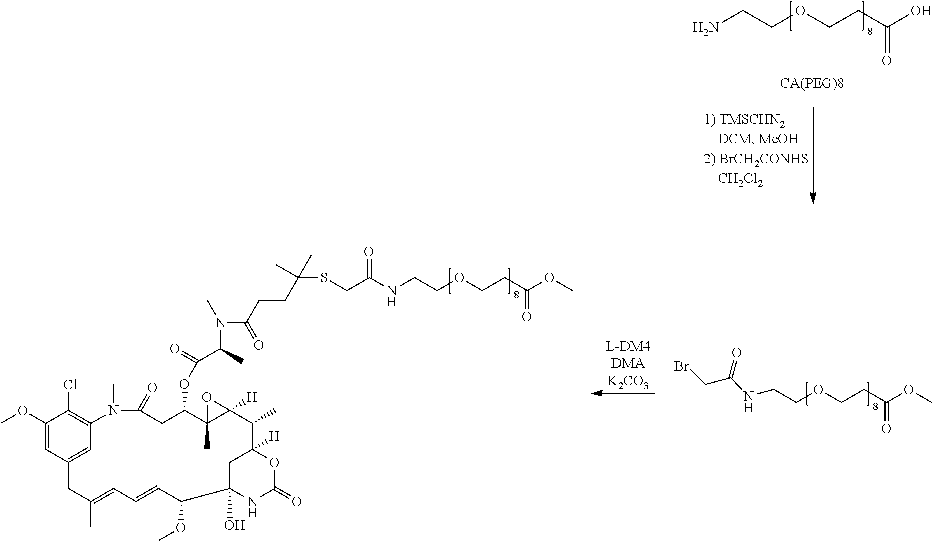

### 13.1. Preparation of Free-Drug L-DM4-AcNH-PEG-COOMe

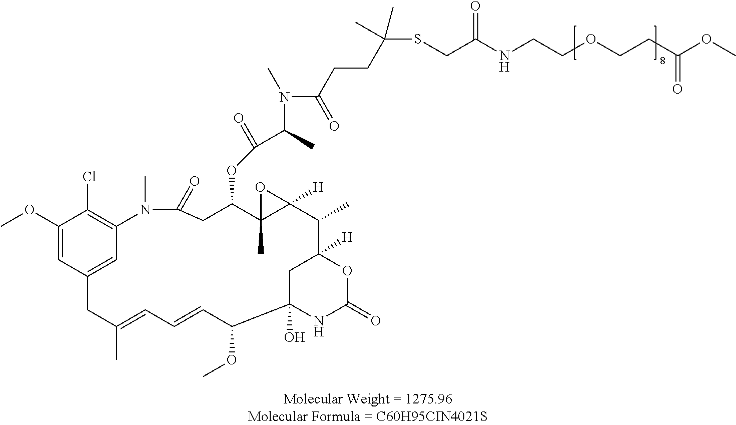

Under magnetic stirring, at RT, in a glass vial, 60 mg of L-DM4, 11.4 mg of potassium carbonate, and a solution of 75 mg of 3-{2-[2-(2-{2-[2-(2-{2-[2-(2-bromo-acetylamino)-ethoxy]-ethoxy}-ethoxy)-ethoxy]-ethoxy}-ethoxy)-ethoxy]-ethoxy}-propionic acid methyl ester in 0.7 ml of DMA are successfully introduced. After 22 hrs at RT, the reaction medium is diluted with 12 ml of water, extracted with 2×10 ml of AcOEt. Organic phases are gathered, dried over MgSO₄, filtered and concentrated to dryness under RP. 50 mg of a colourless oil are obtained, which product is diluted with a minimum amount of DCM and purified by flash-chromatography on 5 g CN-grafted silica gel (gradient of elution n heptane/iPrOH/AcOEt with increasing iPrOH portion). After concentration of fractions containing the expected compound under reduce pressure, the residue is diluted with a minimum amount of DMA and purified by flash chromatography on C18-grafted silica gel (Merck, C18, 5 g, 25-40 μm, 12 ml/min, gradient of elution water:acetonitrile 100:0 to 5:95 by volume). After concentration of fractions containing the expected compound under RP, 3.3 mg of the methyl ester L-DM4-AcNH-PEG₈-COOMe are obtained in the form of a colourless film. Mass spectra (C): t_{R}=0.97 min; [M−H]−+HCOOH: m/z 1319. ¹H NMR (400 MHz, in ppm, chloroform-d): 0.73 (s, 3H); 1.10 to 1.19 (m, 7H); 1.21 (s, 3H); 1.23 (s, 3H); 1.39 (td, J=6.1 and 10.1 Hz, 1H); 1.50 (d, J=13.2 Hz, 1H); 1.57 (s, 3H); 1.71 (m, 1H); 1.85 (m, 1H); 2.11 (dd, J=3.4 and 14.2 Hz, 1H); 2.29 (ddd, J=5.1 and 11.1 and 16.0 Hz, 1H); 2.45 (ddd, J=5.4 and 11.2 and 16.1 Hz, 1H); 2.52 (t, J=6.6 Hz, 3H); 2.79 (s, 3H); 2.82 (m, 1H); 2.90 (m, 1H); 2.96 (d, J=9.3 Hz, 1H); 3.07 (d, J=13.2 Hz, 1H); 3.15 (s, 3H); 3.28 (s, 3H); 3.35 (m, 2H); 3.44 (m, 3H); 3.56 (s, 29H); 3.61 (s, 3H); 3.68 (t, J=6.6 Hz, 2H); 3.91 (s, 3H); 4.20 (t, J=12.0 Hz, 1H); 4.70 (dd, J=3.2 and 12.0 Hz, 1H); 5.35 (q, J=7.0 Hz, 1H); 5.59 (dd, J=9.0 and 15.4 Hz, 1H); 6.13 (s, 1H); 6.35 (dd, J=11.0 and 15.4 Hz, 1H): 6.57 (d, J=1.5 Hz, 1H); 6.67 (d, J=11.2 Hz, 1H); 6.78 (d, J=1.5 Hz, 1H); 6.96 (t, J=5.6 Hz, 1H).

### 13.2. Preparation of 3-{2-[2-(2-{2-[2-(2-{2-[2-(2-bromo-acetylamino)-ethoxy]-ethoxy}-ethoxy-ethoxy]-ethoxy}-ethoxy)-ethoxy]-ethoxy}-propionic acid methyl ester

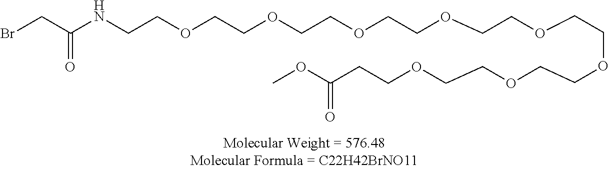

Under magnetic stirring, under an inert atmosphere of Ar, at RT, 200 mg of 3-[2-(2-{2-[2-(2-{2-[2-(2-amino-ethoxy)-ethoxy]-ethoxy}-ethoxy)-ethoxy]-ethoxy}-ethoxy)-ethoxy]-propionic acid (CA(PEG)₈, Pierce), 1.7 ml of DCM, 0.9 ml of MeOH, and 0.34 ml of a 2M trimethylsilydiazomethane solution in hexane are successively introduced in a glass vial. After 30 min at RT, 0.1 ml of a 2M trimethylsilydiazomethane solution in hexane are added. After 25 min at RT, reaction mixture is neutralized by addition of a few drops of acetic acid, concentrated to dryness under RP, azeotroped with toluene. The so obtained colourless oil is diluted with a solution of 106.9 mg of bromo-acetic acid 2,5-dioxo-pyrrolidin-1-yl ester in 0.7 ml of DCM. After 30 min at RT and 16 hrs at 4° C. the crude is purified by flash-chromatography on 20 g CN-grafted silica gel (gradient of elution n.heptane/iPrOH/AcOEt with increasing iPrOH portion). After concentration of fractions containing the expected compound under RP, 175 mg of 3-{2-[2-(2-{2-[2-(2-{2-[2-(2-bromo-acetylamino)-ethoxy]-ethoxy}-ethoxy)-ethoxy]-ethoxy}-ethoxy)-ethoxy]-ethoxy}-propionic acid methyl ester are obtained in the form of a colourless oil. Mass spectra (B): t_{R}=2.79 min; [M+H]+: m/z 576/578.

Bromo-acetic acid 2,5-dioxo-pyrrolidin-1-yl ester could be prepared following published protocol (Biochemistry, 1974, 481).

### Example 14

### L-DM4-Mal-PEG₄-COOMe

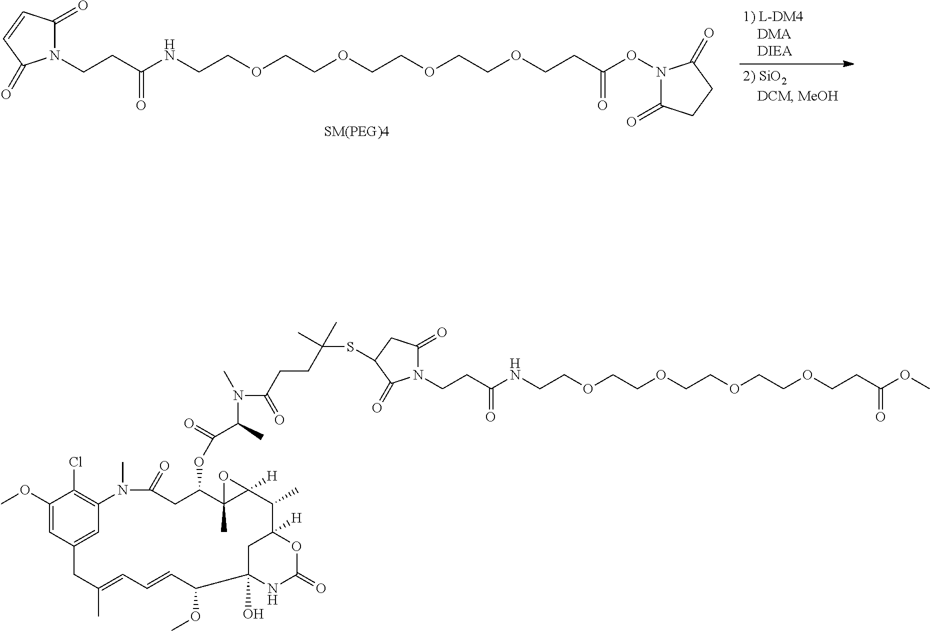

### 14.1. Preparation of free-drug L-DM4-Mal-PEG₄-COOMe

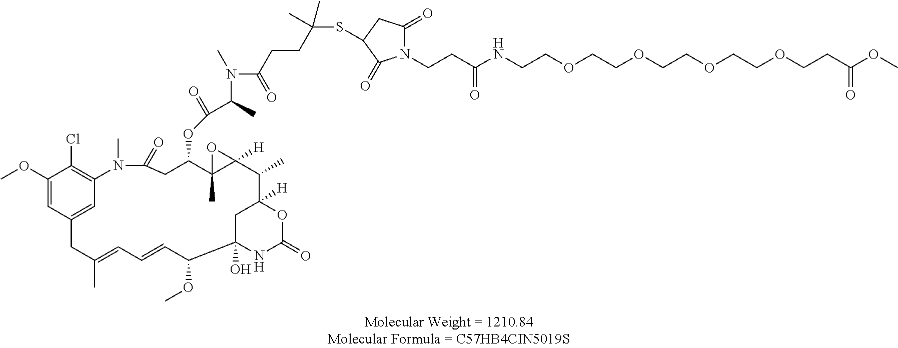

Under magnetic stirring, at RT, 160 mg of L-DM4, 115.8 mg of 3-{2-[2-(2-{2-[3-(2,5-dioxo-2,5-dihydro-pyrrol-1-yl)-propionylamino]-ethoxy}-ethoxy)-ethoxy]-ethoxy}-propionic acid 2,5-dioxo-pyrrolidin-1-yl ester (commercially available, SM(PEG)₄, Pierce), 0.6 ml of DMA, 55.1 mg of supported DIPEA (3.72 mmol/g), 0.3 ml of extra DMA and 6 μl of DIPEA are successively added. After 1 hr at RT and 16 hrs at −20° C., the crude reaction mixture is filtered, washed with DCM, and purified by flash-chromatography on 20 g of silica gel (gradient of elution DCM:MeOH with increasing contribution of MeOH). After concentration of fractions containing the expected product under RP, 110 mg of a colourless film is obtained and purified on 10 g of silica gel (gradient of elution DCM:MeOH with increasing contribution of MeOH). After concentration of fractions containing the expected product under RP, 19.6 mg of the methyl ester L-DM4-Mal-PEG₄-COOMe are obtained in the form of a colourless glass. Mass spectra (A): t_{R}=1.31/1.32 min (2 diastereoisomers); [M−H]−: m/z 1208. ¹H NMR (500 MHz, in ppm, chloroform-d): 0.73 (s, 3H); 1.22 (m, 13H); 1.39 (m, 1H); 1.52 (d, J=13.7 Hz, 1H); 1.57 (s, 3H); 1.78 (m, 1H); 1.99 (m, 1H); 2.11 (ddd, J=1.8 and 1.9 et 14.1 Hz, 1H); 2.24 (ddd, J=4.7 and 11.5 and 15.9 Hz, 1H); 2.38 (m, 3H); 2.46 (m, 1H); 2.53 (m, 3H); 2.69 (s, 1H); 2.82 (s, 3H); 2.94 (dd, J=4.7 and 9.6 Hz, 1H); 3.08 (m, 3H); 3.14 (s, 3H); 3.29 (s, 3H); 3.34 (q, J=5.3 Hz, 2H); 3.43 (d, J=9.1 Hz, 1H); 3.48 (td, J=1.8 and 5.1 Hz, 2H); 3.58 (s, 13H); 3.67 (m, 7H); 3.91 (s, 3H); 4.22 (t, J=11.3 Hz, 1H); 4.70 (m, 1H); 5.29 (m, 1H); 5.59 (m, 1H); 6.30 (s large, 1H); 6.35 (dd, J=11.0 and 15.4 Hz, 1H); 6.61 (m, 2H); 6.76 (s, 1H).

### Example 15

### Inhibition of Growth of MDA-MB-231 and HCT116 Tumor Cells

Cells in exponential phase of growth were trypsinized and resuspended in their respective culture medium (DMEM/F12 Gibco #21331; 10% SVF Gibco #10500-056; 2 nM Glutamine Gibco #25030 for MDA-MB231 & MDA-A1 cells; DMEM (Gibco #11960) 10%, SVF Gibco #10500-056; 2 nM Glutamine Gibco #25030 for HCT116 cells). Cell suspension was distributed in 96-well Cytostar culture plates (GE Healthcare Europe, #RPNQ0163) in complete serum-containing media at a density of 5000 cells/well (MDA-MB231 HCT116). After coating for 4 hrs, serial dilutions were added to triplicate wells. Cells were cultured at 37° C./5% CO₂ in the presence of the drug for 3 days. On the 4^{th} day, 10 μl of a solution of 14C thymidine (0.1 μCi/well (Perkin Elmer #NEC56825000) was added to each well. The uptake of 14C thymidine was measured 96 hrs after the experiment has been started with a microbeta radioactive counter (Perkin Elmer). Cell-free reagent blanks were subtracted from the test well readings and the data were plotted as surviving fractions obtained by dividing readings of the conjugate-treated cells by the average of readings from control wells of vehicle-treated cells.

TABLE III
cellular inhibition proliferation (IC₅₀ in nM)

| example | free-drug | MDA-MB231 | HCT116 |
|---|---|---|---|
| 10 | L-DM4-AcNH-PEG₄- | 15.3 | 13 |
| | COOMe | | |
| 11 | L-DM4-AcNMe-PEG₄- | 28.3 | 18.5 |
| | COOMe | | |
| 12 | L-DM4-Allyl-PEG₄- | 1.5 | 0.8 |
| | COOMe | | |
| 14 | L-DM4-Mal-PEG₄- | 31.7 | 33.6 |
| | COOMe | | |

As is visible, the products tested in the form of esters —COOMe present a strong in vitro proliferation inhibition properties on two different cell lines.

### Example 16

### In Vivo, Evaluation of the Conjugates of hu2H11 and hu2H11R35R74

For the evaluation of anti-tumor activity of conjugates, animals were weighed daily and tumors were measured 2 times weekly by caliper. Tumor weights were calculated using the formula mass (mg)=[length (mm)×width (mm)²]/2. Antitumor activity evaluation was done at the highest non toxic dose (HNTD).

A dosage producing a 20% body weight loss (bwl) at nadir (mean of group) or 10% or more drug deaths, was considered an excessively toxic dosage. Animal body weights included the tumor weights. The primary efficacy end points are ΔT/ΔC, percent median regression, partial and complete regressions (PR and CR) and Tumor free survivors (TFS).

Changes in tumor volume for each treated (T) and control (C) are calculated for each tumor by subtracting the tumor volume on the day of first treatment (staging day) from the tumor volume on the specified observation day. The median ΔT is calculated for the treated group and the median ΔC is calculated for the control group. Then the ratio ΔT/ΔC is calculated and expressed as a percentage;

$$\% Δ T / Δ C = \frac{median ( Tt - T 0 )}{median ( Ct - C 0 )} \times 100$$

The dose is considered as therapeutically active when ΔT/ΔC is lower than 40% and very active when ΔT/ΔC is lower than 10%. If ΔT/ΔC is lower than 0, the dose is considered as highly active and the percentage of regression is dated (ref 1):

% tumor regression: is defined as the % of tumor volume decrease in the treated group at a specified observation day compared to its volume on the first day of first treatment. At a specific time point and for each animal, % regression is calculated. The median % regression is then calculated for the group

$$\% regression ( at t ) = \frac{{volume}_{t 0} - {volume}_{t}}{{volume}_{t 0}} \times 100$$

Partial regression (PR): Regressions are defined as partial if the tumor volume decreases to 50% of the tumor volume at the start of treatment.

Complete regression (CR): Complete regression is achieved when tumor volume=0 mm (CR is considered when tumor volume cannot be recorded).

TFS: Tumor free is defined as the animals with undetectable tumors at the end of the study.

Comparison Between hu2H11-Conjugate and hu2H11R35R74-Conjugate

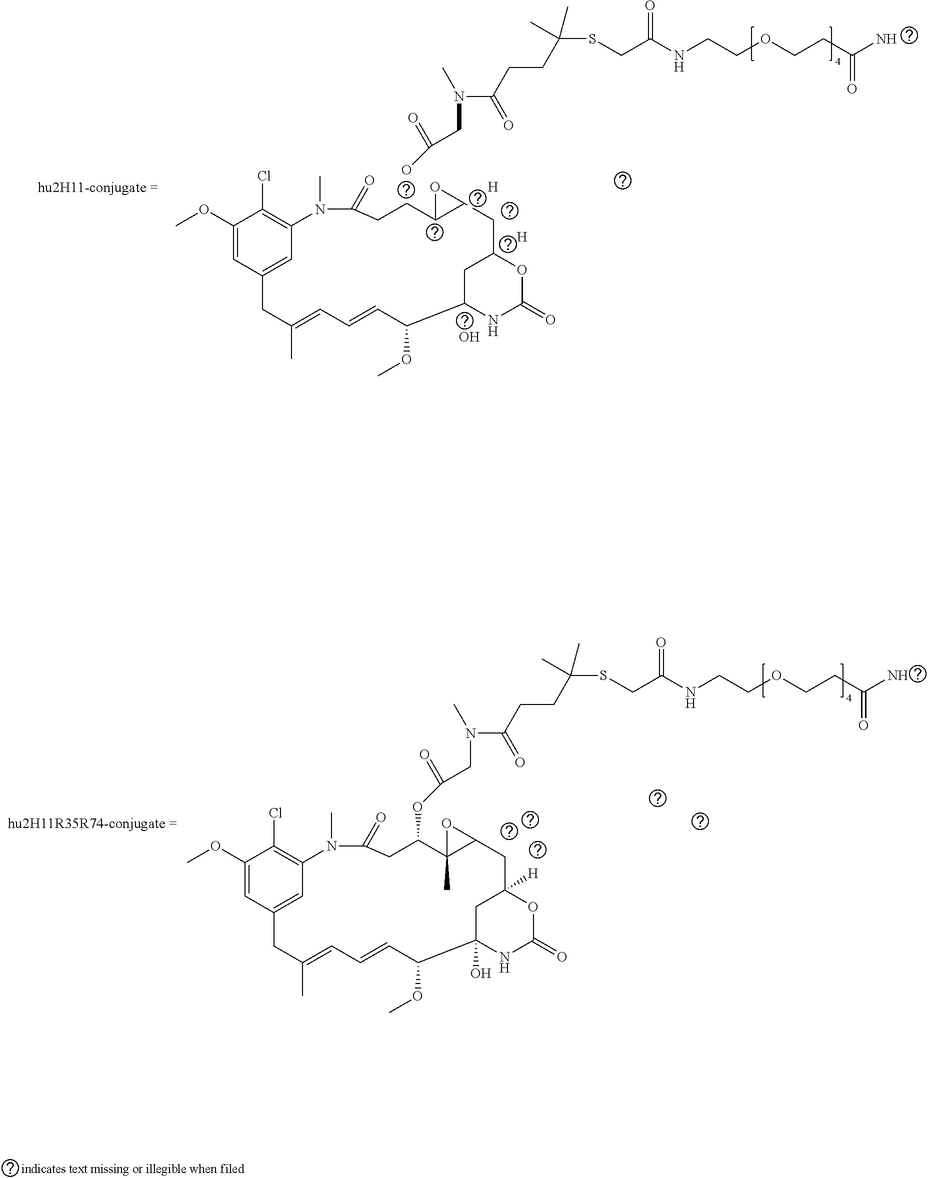

The antitumor activities of hu2h11-conjugate and hu2h11R35R74-conjugate were evaluated at 2 dose levels against a measurable primary colon tumor, CR-LRB-004P, strongly expressing target, S.C. implanted in female SCID mice. Control group was left untreated. Doses were expressed in milligram of protein per kilogram. hu2h11R35R74-conjugate was administered at 40 and 10) mg/kg, by an intravenous (IV) bolus injection, on day 15. To give equivalent dose of DM4, the hu2h11-conjugate was administered at 44 and 11 mg/kg. Results are given in Table IV.

Using a single administration schedule in CR-LRB-004P tumor, hu2h11R35R74-conjugate was active at 40 and 10 mg/kg with a ΔT/ΔC of 28% and 39% respectively while hu2h11-conjugate was active only at 40 mg/kg with a ΔT/ΔC of 26%. At 10 mg/kg, hu2h11-conjugate was not active in this model. From these results, hu2h11R35R74-conjugate at lower dose exhibited a better activity than hu2h11-conjugate.

Conjugate Optimization, Selection of the Optimal Drug Antibody Ratio DAR—Impact of the DAR on the Anti-Tumor Activity of hu2H11R35R74-Conjugate Against Prostatic Adenocarcinoma PC-3 in SCID Female Mice

The effect of the DAR on the antitumor activity of hu2H11R35R74-conjugate was evaluated comparing two low effective doses at six different Drug antibody ratios (DAR) on Prostatic PC-3 tumors S.C. implanted in female SCID. Control group was left untreated. Doses were expressed in milligram of protein per kilogram. DAR was determined by an UV method. hu2H11R35R74-conjugate was administered at 10 and 5 mg/kg with DARs at 3.4, 4.4, 5.9, 6.2, 7.4 and 8.4, respectively, by an intravenous (IV) bolus injection, on day 16. Results are given in Table V.

TABLE IV
Evaluation of the anti-tumor activity of hu2h11-conjugate and hu2h11R35R74-
conjugate against advanced human colon tumor in SCID female mice.

| | | | Dosage in | | Average bwc | | | | | | |
|---|---|---|---|---|---|---|---|---|---|---|---|
| | Route/ | | mg/kg | Drug | in % per | Median | | | | Biosta- | |
| | Dosage in | | protein per | death | mouse at | ΔT/ΔC in % | Regres- | | Tumor free | tistic | |
| | mL/kg per | Schedule | injection | (Day of | nadir (day | day 21 if <0 | sions | | survivors | p value^{a} | |
| Agent | injection | in days | (mg of DM4) | death) | of nadir) | (% regression) | PR | CR | day 30 | Day 21 | Comments |
| hu2h11R35R74- | IV | 15 | 40 (1.6) | 0/6 | −14.7 (25) | 28 | 0/6 | 0/6 | 0/6 | 0.011 | Active |
| conjugate | 16 mL/kg | | 10 (0.4) | 0/6 | −18.2 (25) | 39 | 0/6 | 0/6 | 0/6 | 0.0174 | Active |
| DAR (UV) = | | | | | | | | | | | |
| 5.9 | | | | | | | | | | | |
| hu2h11- | IV | 15 | 44 (1.6) | 0/6 | −13.6 (25) | 26 | 0/6 | 0/6 | 0/6 | 0.0008 | Active |
| conjugate | 16 mL/kg | | 11 (0.4) | 0/6 | −15.8 (25) | 76 | 0/6 | 0/6 | 0/6 | NS | Inactive |
| DAR (UV) = | | | | | | | | | | | |
| 5.3 | | | | | | | | | | | |
| Control | — | — | — | 0/8 | −14.7 (27) | — | 0/8 | 0/8 | 0/8 | | |

TABLE V
Evaluation of the anti-tumor activity of hu2H11R35R74-conjugate at differents DAR* against advanced
human prostatic adenocarcinoma PC-3 SCID female mice (DARs in this Table arc DAR(UV)).

| | | | Dosage in | | Average bwl | | | | | | | |
|---|---|---|---|---|---|---|---|---|---|---|---|---|
| | Route/ | | mg/kg per | | in % per | Median | Median | | | Tumor | Biosta- | |
| | Dosage in | | injection | | mouse at | ΔT/ΔC | % of | Regressions | | free | tistic | |
| | mL/kg per | Schedule | (total | Drug | nadir (day | in % | regression | PR | CR | survivor | p value^{a} | |
| Agent | injection | in days | dose) | death | of nadir) | day 27 | day 27 | Partial | Complete | day 34 | d 27 | Comments |
| DAR = | IV | 16 | 10 | 0/8 | −3.4 (23) | 20 | — | 0/8 | 0/8 | 0/8 | NS | Inactive |
| 3.4 | 16 mL/kg | | 5 | 0/8 | −11.9 (30) | 52 | — | 0/8 | 0/8 | 0/8 | NS | Inactive |
| DAR = | IV | 16 | 10 | 0/8 | −4.4 (17) | <0 | 6.8 | 1/8 | 0/8 | 0/8 | 0.0020 | Active |
| 4.4 | 16 mL/kg | | 5 | 0/8 | −4.5 (25) | 40 | — | 0/8 | 0/8 | 0/8 | NS | Inactive |
| DAR = | IV | 16 | 10 | 0/8 | −5.3 (17) | <0 | 42.8 | 5/8 | 0/8 | 0/8 | <0.0001 | High Activity |
| 5.9 | 16 mL/kg | | 5 | 0/8 | −9.3 (34) | 3 | — | 5/8 | 1/8 | 0/8 | 0.0013 | High Activity |
| DAR = | IV | 16 | 10 | 0/8 | −5.4 (17) | <0 | 13.4 | 2/8 | 0/8 | 0/8 | <0.0001 | High Activity |
| 6.2 | 16 mL/kg | | 5 | 0/8 | −8.0 (34) | 10 | — | 2/8 | 0/8 | 0/8 | 0.0043 | High Activity |
| DAR = | IV | 16 | 10 | 0/8 | −5.0 (17) | <0 | 66 | 6/8 | 0/8 | 0/8 | <0.0001 | High Activity |
| 7.4 | 16 mL/kg | | 5 | 0/8 | −6.3 (17) | <0 | 35.5 | 4/8 | 0/8 | 0/8 | <0.0001 | High Activity |
| DAR = | IV | 16 | 10 | 0/8 | −6.3 (17) | <0 | 59.6 | 7/8 | 2/8 | 0/8 | <0.0001 | High Activity |
| 8.4 | 16 mL/kg | | 5 | 0/8 | −15.6 (34) | 8 | — | 2/8 | 0/8 | 0/8 | 0.0014 | High Activity |
| Control | | | | 0/10 | −19.6 (27) | 100 | 100 | 0/10 | 0/10 | 0/10 | | cachexia |

*each DAR corresponds to a new batch of the conjugate

Using a single administration schedule, hu2H11R35R74-conjugate at 10 mg/kg showed an activity from a DAR of 4.4 to 8.4. At 5 mg/kg, hu2H11R35R74-conjugate showed an activity from a DAR of 5.9 to 4. In conclusion, the DAR has an effect on the tumor activity of hu2H11R35R74-conjugate. From these results on a specific tumor, the DAR(UV) should be above 4. The optimal DAR will be at least equal to 5.9.

### Example 17

### Evaluation of DAR on the PK Parameters of hu2H11R35R74-Conjugate

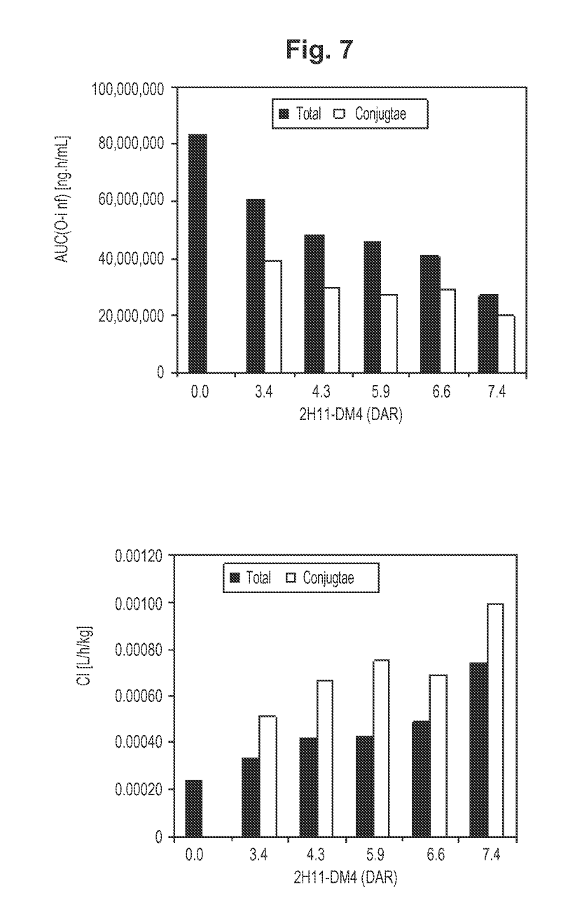

The pharmacokinetic properties of hu2H11R35R74-conjugate at different drug-antibody ratio (DAR) were evaluated in male CD-1 mice after a single intravenous (IV) administration of 20 mg/kg of conjugate. Plasma levels of conjugates were measured to establish basic single dose pharmacokinetic parameters under standard conditions. PK parameters were compared to those of the naked parental antibody. The plasma concentrations of conjugates and their antibody component (total antibody, a sum of conjugated antibody and any de-conjugated antibody) were measured by specific ELISA techniques. Results are given on FIG. 7.

Results showed a reverse correlation between the DAR values and the exposure to the total antibody components with AUC0-∞ values of 83,000,000, 61,000,000, 48,000,000, 46,000,000, 41,000,000 and 27,000.000 ng·h/mL for DAR of 0, 3.4, 4.3, 5.9, 6.6 and 7.4, respectively.

Similarly there is a reverse correlation between the DAR values and the exposure to the conjugate with AUC0-∞ values of 39,000,000, 30,000,000, 27,000,000, 29,000,000 and 20,000,000 ng·h/mL for DAR of 3.4, 4.3, 5.9, 6.6 and 7.4, respectively.

There is a perfect correlation between the DAR values and the elimination of the antibody component with Cl values of 0.00024, 0.00033, 0.00042, 0.00043, 0.00049 and 0.00074 L/h/kg for DAR 0, 3.4, 4.3, 5.9, 6.6 and 7.4, respectively.

Similarly there is almost a perfect correlation between the DAR values and the elimination of the conjugate with Cl values of 0.00051, 0.00066, 0.00075, 0.00069, 0.00099 L/h/kg for DAR 3.4, 4.3, 5.9, 6.6 and 7.4, respectively.

In conclusion, the DAR has an impact on the PK parameters with a decreased exposure and an increased elimination when the DAR increases. According to results from efficacy and PK evaluation, the optimal DAR will be included between 5.9 and 7.4.

### Example 18

### Evaluation of hu2H11R35R74 Conjugate Against Prostatic Adenocarcinoma PC-3 in SCID Female Mice

The antitumor effect of antibody drug conjugate hu2H11R35R74-conjugate having a DAR=5.9 was evaluated at 8 dose levels against measurable prostatic PC-3 tumor, strongly expressing target. S.C. implanted in female SCID mice. Control group was left untreated. Doses were expressed in milligram of protein per kilogram. They were administered at 160, 120, 80, 40, 20, 10, 5 and 2.5 mg/kg, by an intravenous (IV) bolus injection, on day 17. Results are given in Table VI.

Using a single administration schedule, the highest dose of conjugate tested (160 mg/kg) was found to be toxic, inducing body weight loss and drug-related deaths. At the HNTD (120 mg/kg) and other lowest doses, the compound was highly active. For all doses except for 2.5 mg/kg, hu2H11R35R74-conjugate induced partial regressions and for 120, 80 and 20 mg/kg, it induced complete regressions. In addition, the tumor model was cachexic, and the administration of the compound reduced the body weight loss at nadir in comparison with Control. In conclusion, hu2H11R35R74-conjugate showed a high activity with a good dose-effect on Prostatic PC-3 tumor model.

TABLE VI
Evaluation of the anti-tumor activity of hu2H11R35R74 conjugate against advanced human prostatic adenocarcinoma PC-3 SCID female mice.

| | | | | | Average | | | | | | | | |
|---|---|---|---|---|---|---|---|---|---|---|---|---|---|
| | | | Dosage in | | bwc in % | | | | | | | | |
| | Route/ | | mg/kg per | Drug | per mouse | Median | | | | | | | |
| | Dosage in | | injection | death | at nadir | ΔT/ΔC | Median % | Regression: | | TFS | Biostatistic | | |
| Agent | mL/kg per | Schedule | (total | (Day of | (day of | in % | of re- | PR | CR | day | p value^{c} | | |
| (batch) | injection | in days | dose) | death) | nadir) | (day) | gression | (Partial) | (Complete) | 49 | d 26 | d 31 | Comments |
| DAR = | IV | 17 | 160.0 | 1/5 (24) | −21.0 (26) | — | — | | | — | | | Toxic |
| 5.9 | 25 mL/kg | | 120.0 | 0/5 | −14.4 (24) | <0 (31) | 28.4 | 4/5 | 1/5 | 0/5 | NS | <0.0001 | HNTD |
| | | | | | | | | | | | | | High |
| | | | | | | | | | | | | | activity |
| | IV | 17 | 80.0 | 0/5 | −10.3 (24) | <0 (31) | 28.4 | 4/5 | 2/5 | 0/5 | 0.0128 | <0.0001 | High |
| | 16 mL/kg | | | | | | | | | | | | activity |
| | | | 40.0 | 0/6 | −6.9 (20) | <0 (31) | 51.7 | 6/6 | 0/6 | 0/6 | <0.0001 | <0.0001 | High |
| | | | | | | | | | | | | | activity |
| | | | 20.0 | 0/6 | −3.7 (49) | <0 (31) | 53.9 | 5/6 | 1/6 | 0/6 | <0.0001 | <0.0001 | High |
| | | | | | | | | | | | | | activity |
| | | | 10.0 | 0/6 | −10.9 (35) | 19 (26) | — | 1/6 | 0/6 | 0/6 | 0.0002 | 0.0008 | High |
| | | | | | | | | | | | | | activity |
| | | | 5.0 | 0/6 | −2.8 (33) | 3 (26) | — | 1/6 | 0/6 | 0/6 | <0.0001 | 0.0024 | High |
| | | | | | | | | | | | | | activity |
| | | | 2.5 | 0/6 | −9.4 (33) | 5 (26) | — | 0/6 | 0/6 | 0/6 | <0.0001 | NS | Active |
| Control | — | — | — | 0/8 | −29.1 (35) | 100 | | 1/8 [symbol] | 0/8 | 0/8 | | | — |

[symbol]

indicates data missing or illegible when filed

We claim:

1. A compound of formula (I): 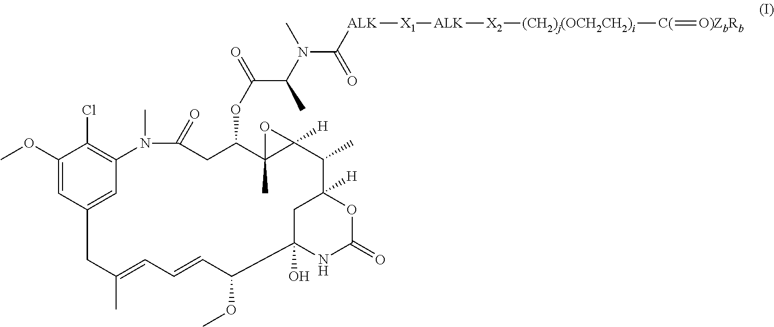
wherein:
ALK is a (C₁-C₆)alkylene group;
X₁ et X₂ are each independently one of the following groups: —CH═CH—, —CO—, —CONR—, —NRCO—, —COO—, —OCO—, —OCONR—, —NRCOO—, —NRCONR′—, —NR—, —S(O)ₙ (n=0.1 or 2) or —O—;
R and R′ are independently H or a (C₁-C₆)alkyl group;
i is an integer of from 1 to 40;
j is an integer corresponding to 1 when X₂ is —CH═CH— and 2 when X₂ is not —CH═CH—;
Z_{b} is a simple bond, —O— or —NH— and R_{b} is H or a (C₁-C₆)alkyl, (C₃-C₇)cycloalkyl, aryl, heteroaryl or (C₃-C₇)heterocycloalkyl group; or Z_{b} is a single bond and R_{b} is Hal.

2. A compound according to claim 1 wherein i is an integer of from 1 to 20.

3. A compound according to claim 1 wherein i is an integer of from 1 to 10.

4. A compound according to claim 1 wherein X₂ is —CH═CH— or —CONR—, the CO group being linked to the —X₁-ALK-group and R being H or a (C₁-C₆)alkyl group.

5. A compound according to claim 1 wherein —X₁-ALK- is —S—CH₂—.

6. A compound according to claim 1 wherein i is 3, 4, 5, 6, 7, 8, 9 or 10.

7. A compound according to claim 1 of formula (II):

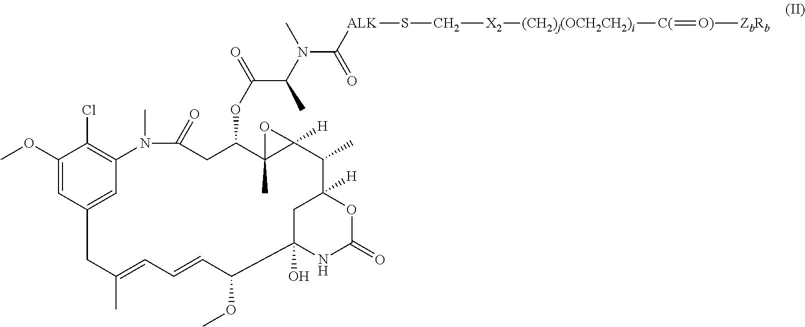

8. A compound selected among the following ones:

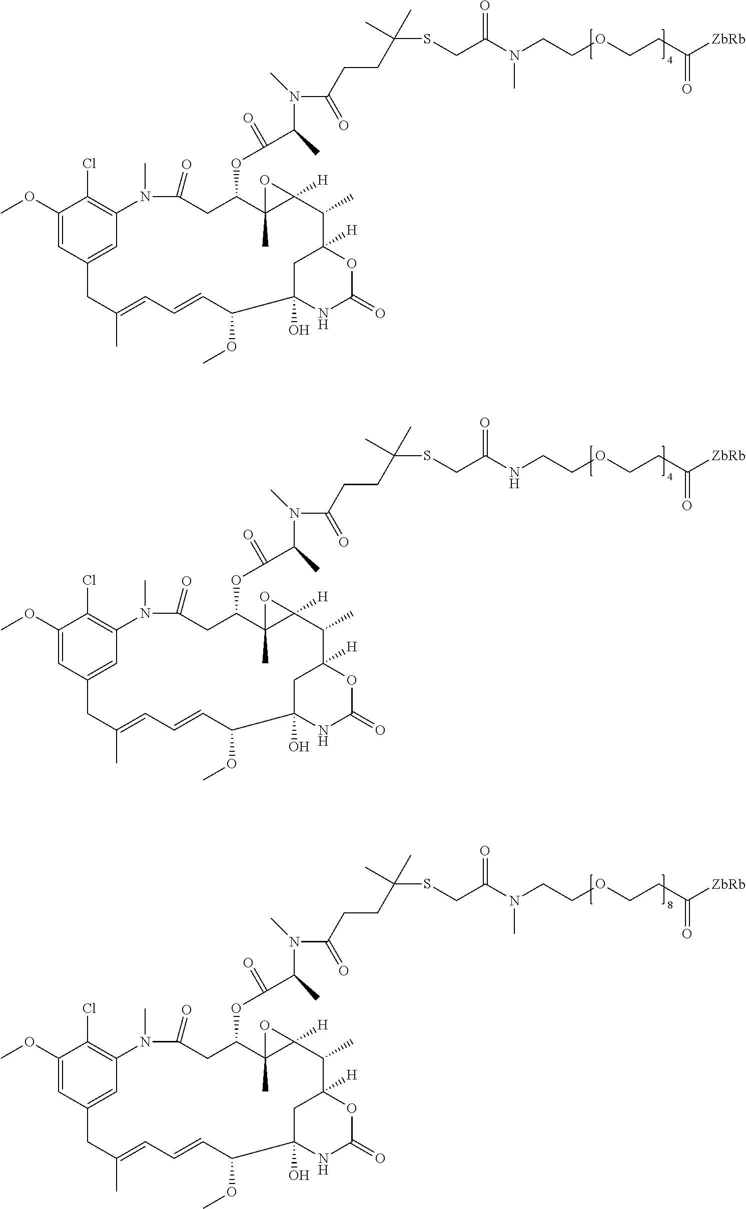

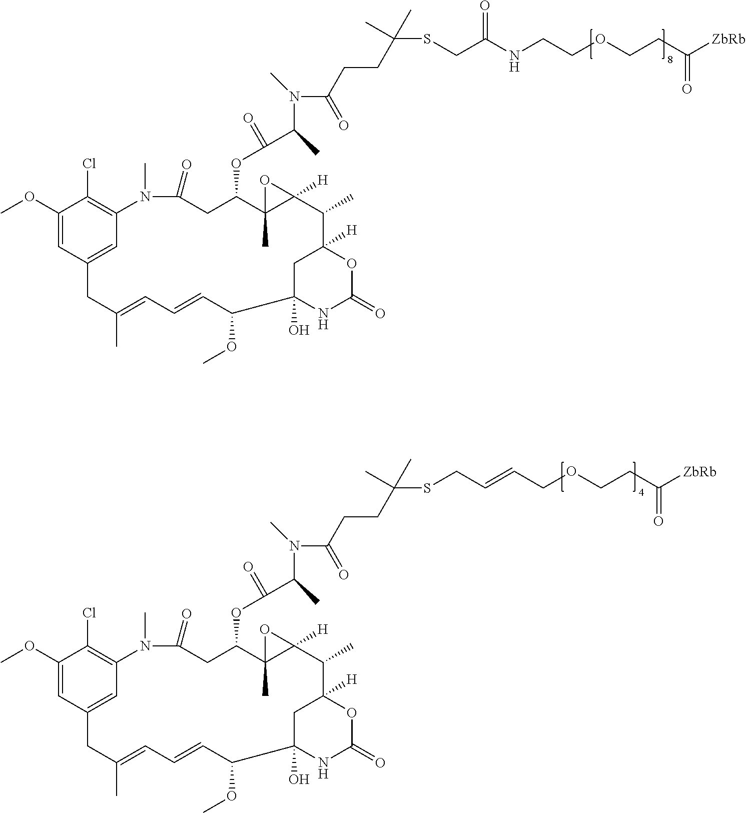

9. A compound according to claim 1 wherein —COZ_{b}R_{b} is —COOH, —COO(C₁-C₆)alkyl, —COOMe, COOCH₂CH═CH₂, 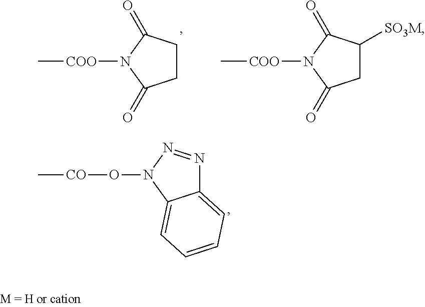
the group 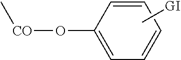
wherein GI represent at least one inductive group NO₂, Hal or F or

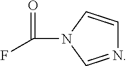

10. A compound according to claim 1 in the form of a base or a salt or a solvate or an hydrate of said base or said salt.

11. A process of preparation of a conjugate comprising the steps of:
(i) bringing into contact an optionally-buffered aqueous solution of an antibody with a solution of a compound according to claim 1;
(ii) then optionally separating the conjugate which was formed in (i) from the unreacted reagents and any aggregate which may be present in the solution.

12. A process according to claim 11 wherein the temperature of the reaction usually varies from 20 to 40° C. and/or the reaction time varies from 1 to 24 hours.

13. A process according to claim 11 wherein after step (i) or (ii), the conjugate-containing solution is submitted to an additional step (iii) of ultrafiltration and/or diafiltration.

14. A process according to claim 11 wherein the antibody is an antibody or an epitope-binding fragment thereof that specifically binds to an EphA2 receptor and comprises at least one heavy chain and at least one light chain, wherein said heavy chain comprises three sequential complementarity-determining regions having amino acid sequences represented by SEQ ID NOS: 1, 2, and 3, and wherein said light chain comprises three sequential complementarity-determining regions having amino acid sequences represented by SEQ ID NOS: 4, 5, and 6.

15. A process according to claim 14 wherein the antibody or the epitope-binding fragment is a humanized or resurfaced antibody or epitope-binding fragment thereof.

16. A process according to claim 14 wherein said heavy chain comprises an amino acid sequence consisting of SEQ ID NO: 12, and wherein said light chain comprises an amino acid sequence consisting of SEQ ID NO: 14.

17. A process according to claim 14 wherein said heavy chain consists in an amino acid sequence SEQ ID NO: 18, and wherein said light chain consists in an amino acid sequence SEQ ID NO: 16.

18. A conjugate obtained by a process according to claim 11.

19. A conjugate according to claim 18 having an average DAR, measured with a UV spectrophotometer, above 4, the DAR being determined by the following equation
DAR=c_{D}/c_{A}
with:
c_{D}=[(ε_{A280}×A₂₅₂)−(ε_{A252}×A₂₈₀)]/[(ε_{D252}×ε_{A280})−(ε_{A252}×ε_{D280})]
c_{A}=[A₂₈₀−(c_{D}×ε_{D280})]/ε_{A280}
ε_{D252}=26,159 M⁻¹cm⁻¹
ε_{D280}=5,180 M⁻¹cm⁻¹
ε_{A280}=224,000 M⁻¹cm⁻¹
ε_{A252}=82,880 M⁻¹cm⁻¹
A₂₅₂ and A₂₈₀ being the absorbances of the conjugate measured on the UV spectrophotometer at respectively 252 and 280 nm.

20. A conjugate according to claim 19 wherein the DAR is between 4 and 10.

21. A conjugate according to claim 20 wherein the DAR is between 4 and 7.

22. A conjugate according to claim 19 wherein the DAR is between 5 and 8.

23. A conjugate according to claim 22 wherein the DAR is between 5.5 and 8

24. A conjugate according to claim 23 wherein the DAR is between 5.9 and 7.5.

25. An aqueous solution comprising a conjugate according to claim 18.

26. A method of treating cancer in a patient in need thereof comprising administering to said patient an effective dose of a compound of claim 1.

27. A method of treating cancer in a patient in need thereof comprising administering to said patient an effective dose of a compound of claim 18.

28. A process for preparing a conjugate comprising covalently linking a compound of claim 1 to an antibody.


SEQUENCE LISTING (18 sequences)

SEQ ID NO: 1 - protein, 5 aa, Homo sapiens
AYYMH

SEQ ID NO: 2 - protein, 17 aa, Mus musculus
LVNPYNGFSSYNQKFQG

SEQ ID NO: 3 - protein, 10 aa, Mus musculus
EFYGYRYFDV

SEQ ID NO: 4 - protein, 16 aa, Mus musculus
KSSQSLIHSDGRTYLN

SEQ ID NO: 5 - protein, 7 aa, Mus musculus
LVSRLDS

SEQ ID NO: 6 - protein, 9 aa, Mus musculus
WQGSHFPRT

SEQ ID NO: 7 - DNA, 357 bp, Mus musculus
  CDS (1)..(357)
gaggtccagctgcaacagtctggacctgagctggtgaagcctggggcttcagtgaagatttcctgcaaggcttctggttactcattcactgcctactacatgcactgggtgaagcaaagtcatgtaaagagtcttgagtggattggacttgttaatccttacaatggttttagtagctacaaccagaatttcgaggacaaggccagcttgactgtagatagattctccagcaccgcctacatggaactccacagcctgacatctgaggactctgcagtctattactgtgcaagagaattctacggctaccggtacttcgatgtctggggcgcagggaccgcggtcaccgtctcctca

SEQ ID NO: 8 - protein, 119 aa, Mus musculus
EVQLQQSGPELVKPGASVKISCKASGYSFTAYYMHWVKQSHVKSLEWIGLVNPYNGFSSYNQNFEDKASLTVDRFSSTAYMELHSLTSEDSAVYYCAREFYGYRYFDVWGAGTAVTVSS

SEQ ID NO: 9 - DNA, 339 bp, Mus musculus
  CDS (1)..(339)
gatgttgtgatgacccagactccactcactttgtcggttaccattggacaaccagcctccatctcttgcaagtcaagtcagagcctcatacatagtgatggaagaacatatttgaattggttgttacagaggccaggccagtctccaaagcgcctaatttatctggtgtctagactggactctggagtccctgacaggttcactggcagtggatcagggacagatttcacactgaaaatcagcagagtggaggctgaggatttgggagtttattattgctggcaaggttcacattttcctcggacgttcggtggaggcaccaagctggaaatcaaacgg

SEQ ID NO: 10 - protein, 113 aa, Mus musculus
DVVMTQTPLTLSVTIGQPASISCKSSQSLIHSDGRTYLNWLLQRPGQSPKRLIYLVSRLDSGVPDRFTGSGSGTDFTLKISRVEAEDLGVYYCWQGSHFPRTFGGGTKLEIKR

SEQ ID NO: 11 - DNA, 357 bp, Homo sapiens
  CDS (1)..(357)
caggtgcaactggtgcaatccggtgccgaggtcgtcaaacccggagcatctgtgaagatatcctgtaaggcctccggctacacttttacagcctactatatgcattgggttaaacagagtcccgtgcagtccctggaatggatcggcttggtgaacccttataacggattctcaagttacaatcaaaagtttcagggcaaggcttccctgactgtagacagatctagttccacagcctacatggagctccattcactgacatcagaagacagcgccgtatactattgcgcacgtgagttctacggctatagatactttgacgtctggggccaaggcacagccgtcacagtgagctct

SEQ ID NO: 12 - protein, 119 aa, Homo sapiens
QVQLVQSGAEVVKPGASVKISCKASGYTFTAYYMHWVKQSPVQSLEWIGLVNPYNGFSSYNQKFQGKASLTVDRSSSTAYMELHSLTSEDSAVYYCAREFYGYRYFDVWGQGTAVTVSS

SEQ ID NO: 13 - DNA, 336 bp, Homo sapiens
  CDS (1)..(336)
gacgtcgtgatgacacaaacccctctgtccctgagcgtcactctgggacaacccgcttccattagctgcaaatcatcacaatctctcatccactcagacggccgtacgtacctcaattggctgctgcagagaccaggacagtcccctaaaaggcttatctacctggtctctcgtttggactctggtgtaccagaccggtttactggttccggggccggaaccgatttcactctgaagatttccagggtggaagctgaagatctcggagtgtattattgctggcagggcagccatttcccccgtacttttggtgggggtaccaaattggaaattaag

SEQ ID NO: 14 - protein, 112 aa, Homo sapiens
DVVMTQTPLSLSVTLGQPASISCKSSQSLIHSDGRTYLNWLLQRPGQSPKRLIYLVSRLDSGVPDRFTGSGAGTDFTLKISRVEAEDLGVYYCWQGSHFPRTFGGGTKLEIK

SEQ ID NO: 15 - DNA, 717 bp, Homo sapiens
  CDS (1)..(717)
atgggatggtcttgcatcatcctgtttctcgtggctactgccaccggagtgcacagtgacgtcgtgatgacacaaacccctctgtccctgagcgtcactctgggacaacccgcttccattagctgcaaatcatcacaatctctcatccactcagacggccgtacgtacctcaattggctgctgcagagaccaggacagtcccctaaaaggcttatctacctggtctctcgtttggactctggtgtaccagaccggtttactggttccggggccggaaccgatttcactctgaagatttccagggtggaagctgaagatctcggagtgtattattgctggcagggcagccatttcccccgtacttttggtgggggtaccaaattggaaattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 16 - protein, 238 aa, Homo sapiens
MGWSCIILFLVATATGVHSDVVMTQTPLSLSVTLGQPASISCKSSQSLIHSDGRTYLNWLLQRPGQSPKRLIYLVSRLDSGVPDRFTGSGAGTDFTLKISRVEAEDLGVYYCWQGSHFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 1404 bp, Homo sapiens
  CDS (1)..(1404)
atgggatggtcctgtattattctgtttctcgtggctacagccacaggcgttcatagtcaggtgcaactggtgcaatccggtgccgaggtcgtcaaacccggagcatctgtgaagatatcctgtaaggcctccggctacacttttacagcctactatatgcattgggttaaacagagtcccgtgcagtccctggaatggatcggcttggtgaacccttataacggattctcaagttacaatcaaaagtttcagggcaaggcttccctgactgtagacagatctagttccacagcctacatggagctccattcactgacatcagaagacagcgccgtatactattgcgcacgtgagttctacggctatagatactttgacgtctggggccaaggcacagccgtcacagtgagctctgcttccactaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 18 - protein, 467 aa, Homo sapiens
MGWSCIILFLVATATGVHSQVQLVQSGAEVVKPGASVKISCKASGYTFTAYYMHWVKQSPVQSLEWIGLVNPYNGFSSYNQKFQGKASLTVDRSSSTAYMELHSLTSEDSAVYYCAREFYGYRYFDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG